(19) United States
(12) Patent Application Publication
(10) Pub. No.: US 2022/0319641 A1
(43) Pub. Date: Oct. 6, 2022

(54) MACHINE-LEARNING MODEL FOR DETECTING A BUBBLE WITHIN A NUCLEOTIDE-SAMPLE SLIDE FOR SEQUENCING

(71) Applicants: Illumina, Inc., San Diego, CA (US); Illumina Software, Inc., San Diego, CA (US)

(72) Inventors: BRANDON TYLER WESTERBERG, San Diego, CA (US); JUNQI YUAN, San Diego, CA (US); ROBERT EZRA LANGLOIS, San Diego, CA (US); MARK DAVID HAHM, Hartland, WI (US); GAVIN DEREK PARNABY, Laguna Niguel, CA (US); THOMAS GROS, Encinitas, CA (US)

(21) Appl. No.: 17/656,173

(22) Filed: Mar. 23, 2022

Related U.S. Application Data
(60) Provisional application No. 63/170,072, filed on Apr. 2, 2021.

(51) Int. Cl. G16B 40/20; C12Q 1/6869; G06N 20/10

(52) U.S. Cl. CPC G16B 40/20; C12Q 1/6869; G06N 20/10

(57) ABSTRACT

Methods, systems, and non-transitory computer readable media are disclosed for accurately and efficiently detect when bubbles impact nucleic-acid-sequencing runs based on data captured during (or derived from) base calls during sequencing runs. In particular, in one or more embodiments, the disclosed systems receive data identifying nucleobase calls and data identifying quality metrics for the nucleobase calls during sequencing cycles. Based on particular nucleobase calls and threshold markers for the quality metrics, the disclosed system utilizes a machine-learning-model to detect a presence of a bubble in a nucleotide-sample slide. Beyond simply detecting the presence of a bubble, the disclosed system can also classify different detected bubbles, such as air bubbles, oil bubbles, or ghost bubbles, or other outputs during sequencing. By utilizing call data and quality metrics, the disclose system can use readily available sequencing data in a platform-agnostic approach to detect bubbles using a uniquely trained machine-learning model.

### CROSS-REFERENCE TO RELATED APPLICATIONS

The present application claims the benefit of and priority to U.S. Provisional Application No. 63/170,072, filed on Apr. 2, 2021. The aforementioned application is hereby incorporated by reference in its entirety.

### BACKGROUND

In recent years, biotechnology firms and research institutions have improved hardware and software platforms for sequencing and analyzing nucleotides. For instance, some existing nucleic-acid-sequencing systems determine individual nucleobases of nucleic-acid sequences by using conventional Sanger sequencing. By contrast, some existing systems determine such nucleobase sequences by performing sequencing-by-synthesis (SBS). By using SBS, existing systems can monitor thousands, tens of thousands, or more nucleic-acid polymers being synthesized in parallel to detect more accurate base calls from a larger base-call dataset and capture other sequencing information. In some cases, existing systems synthesize oligonucleotides in monoclonal colonies within wells of nucleotide-sample slides, such as a flow cell. After a camera captures images of fluorescent tags illuminating colors from nucleobases incorporated into to such oligonucleotides, for instance, some existing systems send image data to a device with sequencing-data-analysis software to analyze the image data for base calls and to determine a nucleobase sequence for a nucleic-acid polymer (e.g., gene coding regions of a nucleic-acid polymer).

Despite these advances in sequencing, existing nucleic-acid-sequencing systems exhibit several technical shortcomings that, for example, inhibit the accuracy and error detection of base calls, require inefficient re-sequencing and re-analysis of nucleotide samples, and limit error detection to specific hardware on a sequencing device. Indeed, existing systems often inaccurately make base calls or capture unreliable image data because the fluids and gases running through sequencing devices or slides can produce irregularities underlying the image data. For instance, a bubble (e.g., air or oil bubble) in a nucleotide-sample slide may interfere with, create noise within, or otherwise cause data quality issues in the data signatures from such image data for base calls. Such bubbles can not only distort the data signatures for base calls but also inhibit or slow down run quality or yield. Despite the problems caused by bubbles, both existing nucleic-acid-sequencing systems and existing sequencing-data-analysis software often lack effective means of detecting bubbles.

Due in part to bubble-caused errors or other sequencing errors, existing nucleic-acid-sequencing systems often inefficiently re-sequence and re-analyze nucleotide samples. In particular, existing systems and software often perform or consume additional processing, computing, storage resources, and time to generate quality data to correct for data impacted by bubble interference. To illustrate, sequencing runs might be subject to a number of problem types, such as failed sequencing reactions, contamination, poor sample loading, or the presence of bubbles. Because existing systems often fail to identify the presence of bubbles or differentiate bubble interference from other errors, such systems often require users to repeat sequencing runs before successfully identifying an issue.

While rudimentary mechanical methods for detecting bubbles have been developed or contemplated, such detection methods are inefficient and can be limited to specific platform types. For instance, existing nucleic-acid-sequencing systems often require additional information about a sequencing run to identify the presence of bubbles or other sources of sequencing errors. More specifically, conventional nucleic-acid-sequencing systems that run fluids through tubing to a cartridge often require additional hardware to capture data indicating the presence of bubbles. For instance, existing systems often require additional tubing cameras, tubing detectors, or other types of sensors. In certain cases, such systems use ultrasonic or capacitive sensing detectors to identify bubbles passing through tubing. But such local hardware on a sequencing device is limited to wet platforms with tubing and requires additional processing, storage, and analytical resources to implement such bubble-detection methods.

Beyond the inefficiencies of existing mechanisms for detecting bubbles in wet sequencing platforms, some such bubble-detection methods are limited to specific hardware on a sequencing device. As mentioned, some conventional nucleic-acid-sequencing systems attempt to detect bubbles by utilizing hardware-based bubble detectors. Even though some conventional nucleic-acid-sequencing systems could include sensors in tubing or other components to detect bubbles, such detection hardware is not only costly but also infeasible in dry sequencing platforms. For example, dry sequencing platforms often perform fluidics operations on single-use consumables that lack tubing funneling fluids into the consumables. Such dry sequencing platforms either cannot utilize dedicated bubble detection sensors or such sensors would be impractical by requiring a bulky redesign of costly sequencing devices or consumable nucleotide-sample slides.

### BRIEF SUMMARY

This disclosure describes one or more embodiments of systems, methods, and non-transitory computer readable storage media that provide benefits and/or solve one or more of the above problems in the art. For example, the disclosed systems use a machine-learning model to accurately and efficiently detect when bubbles impact nucleic-acid-sequencing runs based on data captured during (or derived from) base calls during such sequencing runs. To illustrate, the disclosed systems can receive data identifying nucleobase calls and data identifying quality metrics for such nucleobase calls from sequencing platforms during sequencing cycles. Based on particular nucleobase calls and threshold markers for the quality metrics, a machine-learning model can detect a presence of a bubble in a nucleotide-sample slide. By using call data and quality metrics, the disclosed system can use readily available sequencing data in a platform-agnostic approach to detect bubbles using a uniquely trained machine-learning model.

In some cases, the disclosed systems use a machine learning model trained to identify bubbles within particular sections or units (e.g., tiles) of a nucleotide-sample slide (e.g., flow cells) during a sequencing cycle. Beyond simply detecting the presence of a bubble, in some examples, the disclosed system can also classify different detected bubbles, such as oil bubbles, air bubbles, or ghost bubbles, or other identify other outputs during sequencing, such as tile registration failures and dropped tiles.

Additional features and advantages of one or more embodiments of the present disclosure will be set forth in the description which follows, and in part will be obvious from the description, or may be learned by the practice of such example embodiments.

### BRIEF DESCRIPTION OF THE DRAWINGS

Various embodiments will be described and explained with additional specificity and detail through the use of the accompanying drawings, which are summarized below.

FIG. 1 illustrates an environment in which a bubble-detection system can operate in accordance with one or more embodiments of the present disclosure.

FIG. 2 illustrates an overview diagram of the bubble-detection system detecting a presence of a bubble in accordance with one or more embodiments of the present disclosure.

FIG. 3 illustrates an overview diagram of the bubble-detection system operating with respect to one-channel, two-channel, and four-channel sequencing data in accordance with one or more embodiments of the present disclosure.

FIGS. 4A-4C illustrate example charts that graph data signatures corresponding to different error classifications in accordance with one or more embodiments of the present disclosure.

FIG. 5 illustrates an example bubble-detection-machine-learning model in accordance with one or more embodiments of the present disclosure.

FIGS. 6A-6C illustrate the bubble-detection system training a bubble-detection-machine-learning model and example spatial images with bubbles within a flow cell in accordance with one or more embodiments.

FIG. 7 illustrates a series of acts for detecting a presence of a bubble in accordance with one or more embodiments of the present disclosure.

FIG. 8 illustrates a block diagram of an example computing device in accordance with one or more embodiments of the present disclosure.

### DETAILED DESCRIPTION

This disclosure describes one or more embodiments of a bubble-detection system that utilizes a machine-learning model to detect the presence of a bubble within nucleotide-sample slides based on data captured during (or derived from) nucleic-acid-sequencing runs. In some embodiments, for instance, the bubble-detection system accesses or receives base-call data for nucleobase calls during sequencing cycles and quality data identifying quality metrics that estimate errors of such nucleobase calls during the sequencing cycles. Such call data and quality data can be specific to a nucleotide-sample slide, such as a flow cell, or a section of the slide. From the call data and quality data, the bubble-detection system determines a subgroup of the nucleobase calls corresponding to at least one nucleobase (e.g., subgroups of adenine and guanine base calls) and a subgroup of the nucleotide calls that meet a threshold quality value. Based on these subgroups of data as inputs, the bubble-detection system utilizes a machine learning model to detect the presence of a bubble within a nucleotide-sample slide. In some such embodiments, such a bubble-detection-machine-learning model classifies a type of bubble detected.

As just indicated, in some embodiments, the bubble-detection system receives call data comprising nucleobase calls for cycles of sequencing a nucleic-acid polymer. Generally, the bubble-detection system receives call data that identifies nucleobases at each sequencing cycle. The bubble-detection system can receive the call data organized or packaged according to various types of data. For instance, the bubble-detection system can receive the call data organized according to on one-channel data, two-channel data, or four-channel data. In any case, the bubble-detection system can receive and utilize call data from various types of sequencing platforms.

As further noted above, the bubble-detection system also receives quality data comprising quality metrics that estimate errors in the nucleobase calls for cycles. In some embodiments, the quality metrics indicate the base call accuracy for the nucleotide-sample slide. For instance, quality metrics can comprise a value indicating the probability of an incorrect base call. In one or more embodiments, a quality metric comprises a quality score (or Q score) that indicates a probability of an incorrect base call for a section of the nucleotide-sample slide is 1 in 100 for a Q20 score, 1 in 1,000 for a Q30 score, 1 in 10,000 for a Q40 score, etc. But the bubble-detection system flexibly receives any number of quality metrics as part of determining the presence of a bubble.

Based on the call data, in some embodiments, the bubble-detection system determines a subset of the nucleobase calls corresponding to at least one nucleobase. For instance, in certain implementations, the bubble-detection system determines a proportion of adenine calls, thymine calls, cytosine calls, or guanine calls. In one example, the bubble-detection system determines a proportion or percentage of base calls in each cycle that comprise adenine calls and a proportion or percentage of base calls in each cycle that comprise thymine calls. Accordingly, in certain implementations, the bubble-detection system determines a percentage (or other subset) of nucleobase calls corresponding to adenine and a percentage (or other subset) of nucleobase calls corresponding to guanine within a particular section of a nucleotide-sample slide.

Based on the quality data, in certain cases, the bubble-detection system can also determine a subset of the nucleobase calls satisfying a threshold quality metric for the quality metrics. In some embodiments, the bubble-detection system determines a threshold quality metric. For example, the bubble-detection system might determine that the threshold quality metric for base calls in a cycle equals Q30 and corresponds with a 99.9% accuracy or a 1 in 1,000 chance that a given base call is incorrect. The bubble-detection system further determines a proportion or percentage of base calls that meet the determined threshold quality metric. In particular, the bubble-detection system compares the quality metrics from the received quality data to the threshold quality metric. Accordingly, in certain implementations, the bubble-detection system determines a percentage (or other subset) of nucleobase calls that satisfy a threshold quality metric within a particular section of a nucleotide-sample slide.

Having determined relevant subsets of nucleobase calls, in certain cases, the bubble-detection system generates an input matrix for a bubble-detection-machine-learning model comprising a first subset of nucleobase calls corresponding to at least one nucleobase and a second subset of nucleobase calls satisfying a threshold quality metric. More specifically, in one example, the bubble-detection system compiles an input matrix using a subset of adenine calls, a subset of guanine calls, and the subset of nucleobase calls that satisfy the threshold quality metric (e.g., for each cycle within a total number of sequencing cycles). The bubble-detection system can accommodate various input sizes by adjusting the input matrix based on the number of sequencing cycles. For example, in one embodiment, the input matrix comprises three one-dimensional input channels of length N where the three input channels comprise the subset of adenine calls, the subset of guanine calls, and the second subset of nucleobase calls that satisfy the threshold quality metric, and N equals the number of sequencing cycles.

Regardless of the input form, the bubble-detection system can use a bubble-detection-machine-learning model to detect a presence of a bubble within the nucleotide-sample slide based on subsets of call data and quality data. To detect the presence of such bubbles, the bubble-detection system can utilize various types of machine learning models. For example, in some embodiments, the bubble-detection system utilizes a neural network, such as a Convolutional Neural Network (CNN), to detect bubbles. In other embodiments, the bubble-detection system utilizes other types of machine learning models to detect bubbles. For instance, in some implementations, the bubble-detection system implements a Support Vector Machine (SVM) or an Adaptive Boosting machine learning model.

As suggested above, the bubble-detection system provides several technical benefits and technical improvements relative to conventional nucleic-acid-sequencing systems and corresponding sequencing-data-analysis software. In particular, the bubble-detection system can improve the accuracy with which existing nucleic-acid-sequencing systems or corresponding software detect the presence of bubbles interfering with sequencing. The disclosed bubble-detection system introduces a first-of-its-kind machine-learning model that detects bubbles within a nucleotide-sample slide unmatched by the state or the art. As noted above, existing systems either cannot directly detect bubbles interfering with sequencing or use mechanical sensors to detect bubbles limited to specific platforms. Unlike such existing systems, the disclosed bubble-detection system utilizes a machine-learning model trained to accurately detect bubbles within a nucleotide-sample slide based on a unique analysis of available data—that is, call data identifying nucleobase calls and quality data identifying quality metrics for such nucleobase calls. By relying on call data and quality data, the bubble-detection system can accurately detect a presence of (and sometimes identify a type of) bubble within a nucleotide-sample slide utilizing the trained bubble-detection-machine-learning model. Unlike conventional and mechanical bubble-detection methods, the bubble-detection system can apply its machine-learning model across various sequencing platforms by using readily available call data and quality data.

In addition to a new and accurate bubble-detection method, in some embodiments, the bubble-detection system can accurately detect a presence of a bubble within a specific section of a nucleotide-sample slide (e.g., within a tile of a flow cell or a group of tiles of a flow cell) and corresponding call data affected by the bubble. More specifically, in certain cases, the bubble-detection system utilizes a bubble-detection-machine learning model that passes call data and quality data specific for slide sections to automatically detect sections of a nucleotide-sample slide that are impacted by bubbles. By specifying which sections of the nucleotide-sample slide have been impacted, the bubble-detection system can excise inaccurate data and improve the accuracy and overall quality of sequencing data. To illustrate, in some implementations, the bubble-detection system either removes reads for a section of a nucleotide-sample slide from the call data or reduces a quality metric for reads or nucleobase calls corresponding to a particular section of a nucleotide-sample slide affected by a bubble. In some cases, the bubble-detection system removes nucleobase calls or reduces a quality metric when a detected bubble equals or exceeds a size threshold or when a data signature for nucleobase calls differs form a norm by a particular threshold.

In addition to improved accuracy, the bubble-detection system improves the efficiency with which conventional nucleic-acid-sequencing systems and corresponding sequencing-data-analysis software determine nucleobase sequences for nucleic-acid polymers. By identifying when bubbles impact or otherwise interfere with the nucleotide-sample slide, the bubble-detection system obviates the need to trouble shoot certain errors and consequently run and re-run multiple sequencing cycles to achieve high quality data. In some such cases, the bubble-detection system identifies a particular section of a nucleotide-sample slide impacted by a bubble to identify with specificity which corresponding portion of data is corrupted or interfered with by the bubble. Furthermore, the bubble-detection system can also improve the efficiency of sequencing by classifying specific types of bubbles (e.g., oil, air, or ghost) or other specific error types for correction (e.g., tile registration failures or dropped tiles). Thus, the bubble-detection system improves the efficiency of sequencing nucleic-acid polymers by recognizing and minimizing data for a section of a nucleotide-sample slide or the number of cycles that need to be discarded or reevaluated to accurately sequence a nucleic-acid polymer.

Beyond reducing re-sequencing efforts or identifying specific bubble-impacted data, in some embodiments, the bubble-detection system improves efficiency relative to conventional nucleic-acid-sequencing systems and corresponding sequencing-data-analysis software by reducing the resources typically required to identify bubbles within sequencing runs. As mentioned previously, the bubble-detection system utilizes a bubble-detection-machine learning model to detect bubbles within sequencing runs. In at least one embodiment, the bubble-detection system utilizes a lightweight CNN identify the presence of bubbles. Thus, instead of requiring the use of additional hardware on a sequencing device (e.g., tubing sensors) or using a computationally heavy neural network to process additional information, in some embodiments, the bubble-detection system more efficiently utilizes a computationally lightweight machine learning model to analyze available call data and quality data from various sequencing platforms. Thus, in such cases, the bubble-detection system creates a low data footprint compared to using images or other sensor data to detect bubbles.

Independent of improved efficiency, the bubble-detection system also improves the flexibility with which nucleic-acid-sequencing systems and corresponding sequencing-data-analysis software detect bubbles. As noted above, in some implementations, the bubble-detection system is platform agnostic and free of additional tube sensors—like those on some fluid-based sequencing devices. In particular, the bubble-detection system flexibly utilizes base call and quality data that is readily accessible from numerous sequencing platforms. In at least one embodiment, the bubble-detection system utilizes a CNN with an adaptive max pooling layer that enables the bubble-detection system to more flexibly analyze variable input sizes. Thus, the bubble-detection system can be implemented and utilized by existing sequencing platforms without the requirement for additional hardware. Furthermore, in some embodiments, the bubble-detection system is flexibly applied utilizing various configurable circuits, such as an Application Specific Integrated Circuit (ASIC) or a Field Programmable Gate Array (FPGA).

As illustrated by the foregoing discussion, the present disclosure utilizes a variety of terms to describe features and advantages of the bubble-detection system. Additional detail is now provided regarding the meaning of such terms. For example, as used herein, the term “nucleotide-sample slide” refers to a plate or slide comprising oligonucleotides for sequencing nucleotide segments for samples. In some embodiments, a nucleotide-sample slide comprises a slide containing fluidic channels through which reagents and buffers can travel as part of a sequencing. For example, in one or more embodiments, the nucleotide-sample slide comprises a flow cell comprising small fluidic channels and short oligonucleotides complementary to adaptor sequences.

As used herein, the term “call data” refers to image data or other digital information indicating individual nucleobases or the sequence of nucleobases for a nucleic-acid polymer. In particular, call data can include intensity values (e.g., color or light intensity values for individual clusters) from images taken by a camera of a nucleotide-sample slide or other data that indicate individual nucleobases or the sequence of nucleobases for a nucleic-acid polymer. In addition or in the alternative to intensity values, the call data may include chromatogram peaks or electrical current changes indicating individual nucleobases in a sequence. Additionally, in some embodiments, call data includes individual nucleobase calls identifying the individual nucleobases (e.g., A, T, C, or G). For example, call data can comprise data for nucleobase calls in a sequence for a nucleic-acid polymer, the number of nucleobase calls corresponding to a particular base (e.g., adenine, cytosine, thymine, or guanine). In some embodiments, call data comprises information from a sequencing device that utilizes sequencing by synthesis (SBS).

As used herein, the term “nucleobase call” refers to an assignment or determination of a particular nucleobase to add to or incorporate within an oligonucleotide for a sequencing cycle. In particular, a nucleobase call indicates an assignment or a determination of the type of nucleotide that has been incorporated within an oligonucleotide on a nucleotide-sample slide. In some cases, a nucleobase call includes an assignment or determination of a nucleobase to intensity values resulting from nucleotides added to an oligonucleotide in a nanowell of a nucleotide-sample slide. Alternatively, a nucleobase call includes an assignment or determination of a nucleobase to chromatogram peaks or electrical current changes resulting from nucleotides passing through a nanopore of a nucleotide-sample slide. By using nucleobase calls, a sequencing system determines a sequence of a nucleic-acid polymer. For example, a single nucleobase call can comprise an adenine call, a cytosine call, a guanine call, or a thymine call.

As further used herein, the term “sequencing cycle” or simply “cycle” refers to an iteration of adding or incorporating a nucleobase to an oligonucleotide or an iteration of adding or incorporating nucleobases to oligonucleotides in parallel. In particular, a cycle can include an iteration of taking an analyzing one or more images with data indicating individual nucleobases added or incorporated into an oligonucleotide or to oligonucleotides in parallel. Accordingly, cycles can be repeated as part of sequencing a nucleic-acid polymer. For example, in one or more embodiments, each sequencing cycle involves either single reads in which DNA or RNA strands are read in only a single direction or paired-end reads in which DNA or RNA strands are read from both ends. Further, in certain cases, each sequencing cycle involves a camera taking an image of the nucleotide-sample slide or multiple sections of the nucleotide-sample slide to generate image data for determining a particular nucleobase added or incorporated into particular oligonucleotides. Following the image capture stage, a sequencing system can remove certain fluorescent labels from incorporated nucleobases and perform another sequencing cycle until the nucleic-acid polymer has been completely sequenced. In one or more embodiments, “cycle” refers to a sequencing cycle within a Sequencing By Synthesis (SBS) run.

As used herein, the term “nucleic-acid polymer” refers to a macromolecule made up of units of nucleic acids. In particular, a nucleic-acid polymer can include a macromolecule composed of different nitrogenous heterocyclic bases in a sequence. For example, a nucleic-acid polymer can include a segment or molecule of deoxyribonucleic acid (DNA), ribonucleic acid (RNA), or other polymeric forms of nucleic acids or chimeric or hybrid forms of nucleic acids noted below. More specifically, in some cases, the nucleic-acid polymer is one found in a sample prepared or isolated by a kit and received by a sequencing device.

As used herein, the term “quality data” refers to information indicating the accuracy or quality of nucleobase calls for a sequencing cycle. In particular, quality data generally indicates the accuracy of one or more base calls within a sequencing cycle. For instance, quality data can comprise one or more quality metrics.

As used herein, the term “quality metric” refers to a specific score or other measurement indicating the accuracy of nucleobase calls for a sequencing cycle. In particular, a quality metric comprises a value indicating the likelihood that one or more predicted nucleobase calls contain errors. For example, in certain implementations, a quality metric can comprise a Q score predicting the error probability of any given base call within a sequencing cycle.

As used herein, the term “bubble” refers to a spherical shaped or sphere-like globule or other container enclosing a gas, liquid, or other material. In particular, a bubble refers to a spherical globule that can enter a nucleotide-sample slide and that can affect the data quality of a sequencing cycle. For example, a bubble can comprise an air bubble or an oil bubble that occurs within a nucleotide-sample slide.

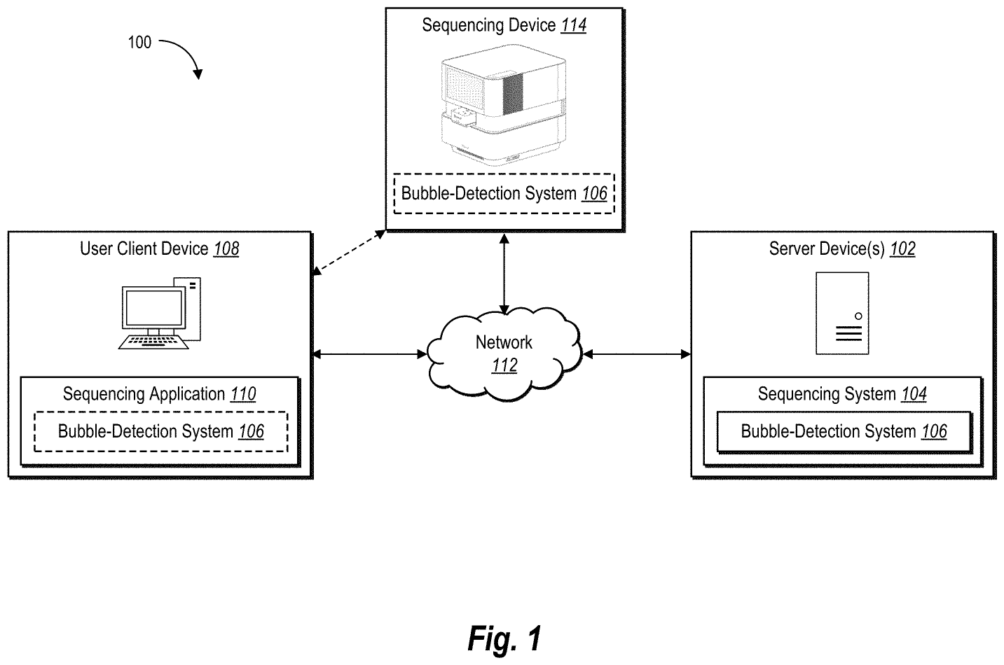

Additional detail will now be provided regarding the bubble-detection system in relation to illustrative figures portraying example embodiments and implementations of the bubble-detection system. For example, FIG. 1 illustrates a schematic diagram of a system environment (or “environment”)100 in which a bubble-detection system 106 operates in accordance with one or more embodiments. As illustrated, the environment 100 includes one or more server device(s) 102 connected to a user client device 108 and a sequencing device 114 via a network 112. While FIG. 1 shows an embodiment of the bubble-detection system 106, alternative embodiments and configurations are possible.

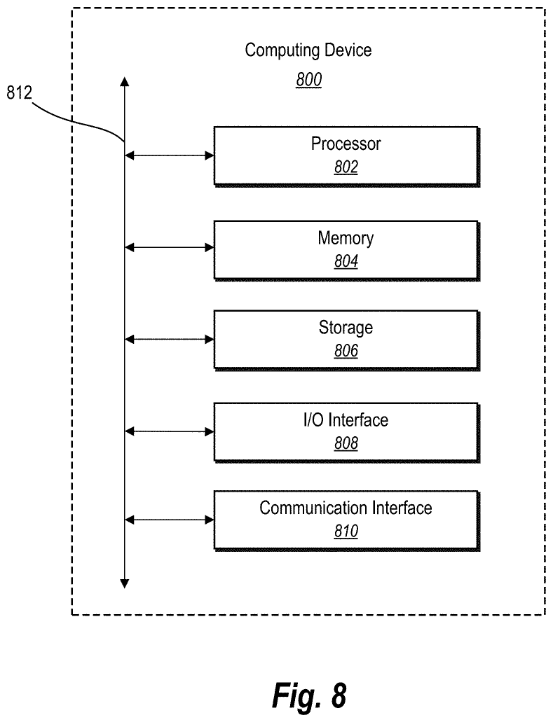

As shown in FIG. 1, the server device(s) 102 the user client device 108, and the sequencing device 114 are connected via the network 112. Accordingly, each of the components of the environment 100 can communicate via the network 112. The network 112 comprises any suitable network over which computing devices can communicate. Example networks are discussed in additional detail below in relation to FIG. 8.

As indicated by FIG. 1, the sequencing device 114 comprises a device for sequencing a nucleic-acid polymer. In some embodiments, the sequencing device 114 analyzes nucleic-acid segments extracted from samples to generate data utilizing computer implemented methods and systems described herein either directly or indirectly on the sequencing device 114. More particularly, the sequencing device 114 receives and analyzes, within nucleotide-sample slides, nucleic-acid segments extracted from samples. In one or more embodiments, the sequencing device 114 utilizes SBS to sequence nucleic-acid polymers. In addition or in the alternative to communicating across the network 112, in some embodiments, the sequencing device 114 bypasses the network 112 and communicates directly with the user client device 108.

As further indicated by FIG. 1, the server device(s) 102 may generate, receive, analyze, store, receive, and transmit electronic data, such as data for determining nucleobase calls or sequencing nucleic-acid polymers. As shown in FIG. 1, the server device(s) 102 may receive data from the sequencing device 114. For example, the server device(s) 102 may gather and/or receive sequencing data including call data, quality data, and other data relevant to sequencing nucleic-acid polymers. The server device(s) 102 may also communicate with the user client device 108. In particular, the server device(s) 102 can send nucleobase sequences, error data, and other information to the user client device 108.

In some embodiments, the server device(s) 102 comprises a distributed server where the server device(s) 102 include a number of server devices distributed across the network 112 and located in different physical locations. The server device(s) 102 can comprise a content server, an application server, a communication server, a web-hosting server, or another type of server.

As further shown in FIG. 1, the server device(s) 102 can include the sequencing system 104. Generally, the sequencing system 104 analyzes sequencing data received from the sequencing device 114 to determine nucleobase sequences for nucleic-acid polymers. For example, the sequencing system 104 can receive raw data from the sequencing device 114 and determine a nucleobase sequence for a nucleic-acid segment. In some embodiments, the sequencing system 104 determines the sequences of nucleobases in DNA and/or RNA segments. In addition to processing and determining sequences for nucleic-acid polymers, the sequencing system 104 also analyzes sequencing data to detect irregularities in sequencing cycles. In particular, the sequencing system 104 can use the bubble-detection system 106 to detect bubbles within a sequencing cycle and send a corresponding notification to the user client device 108.

As just mentioned, and as illustrated in FIG. 1, the bubble-detection system 106 analyzes data from the sequencing device 114 to detect the presence of a bubble within a nucleotide-sample slide associated with the sequencing device 114. More specifically, in some embodiments, the bubble-detection system 106 receives call data and quality data from the sequencing device 114. Based on the call data and the quality data, the bubble-detection system 106 determines a first subset of nucleobase calls corresponding to at least one nucleobase and a second subset of the nucleobase calls satisfying a threshold quality metric. Based on the first subset of the nucleobase calls and the second subset of the nucleobase calls, the bubble-detection system 106 implements a bubble-detection-machine-learning model to detect the presence of a bubble. Accordingly, the bubble-detection system 106 can include one or more machine learning models (e.g., neural networks, SVM, Adaptive Boosting).

As further illustrated and indicated in FIG. 1, the user client device 108 can generate, store, receive, and send digital data. In particular, the user client device 108 can receive sequencing data from the sequencing device 114. Furthermore, the user client device 108 may communicate with the server device(s) 102 to receive nucleobase sequences as well as reports of irregularities within a sequencing cycle, such as alerts indicating the presence of a bubble. The user client device 108 can accordingly present sequencing data and notifications of bubbles within a graphical user interface to a user associated with the user client device 108.

The user client device 108 illustrated in FIG. 1 may comprise various types of client devices. For example, in some embodiments, the user client device 108 includes non-mobile devices, such as desktop computers or servers, or other types of client devices. In yet other embodiments, the user client device 108 includes mobile devices, such as laptops, tablets, mobile telephones, or smartphones. Additional details with regard to the user client device 108 are discussed below with respect to FIG. 8.

As illustrated in FIG. 1, the user client device 108 includes a sequencing application 110. The sequencing application 110 may be a web application or a native application stored and executed on the user client device 108 (e.g., a mobile application, desktop application). The sequencing application 110 can receive data from the bubble-detection system 106 and can present, for display at the user client device 108, sequencing data. Furthermore, the sequencing application 110 can provide a notification indicating the presence of a bubble within a section of a nucleotide-sample slide.

As further illustrated in FIG. 1, the bubble-detection system 106 may be located on the user client device 108 as part of the sequencing application 110. As illustrated, in some embodiments, the bubble-detection system 106 is implemented by (e.g., located entirely or in part) on the user client device 108. Additionally, or alternatively, in some implementations, the bubble-detection system 106 is implemented by (e.g., located entirely or in part) on the sequencing device 114. In yet other embodiments, the bubble-detection system 106 is implemented by one or more other components of the environment 100. In particular, the bubble-detection system 106 can be implemented in a variety of different ways across the server device(s) 102, the network 112, the user client device 108 and the sequencing device 114.

Though FIG. 1 illustrates the components of environment 100 communicating via the network 112, in certain implementations, the components of environment 100 can also communicate directly with each other, bypassing the network. For instance, and as previously mentioned, the user client device 108 can communicate directly with the sequencing device 114. Additionally, the user client device 108 can communicate directly with the bubble-detection system 106. Moreover, the bubble-detection system 106 can access one or more databases housed on or accessed by the server device(s) 102 or elsewhere in the environment 100.

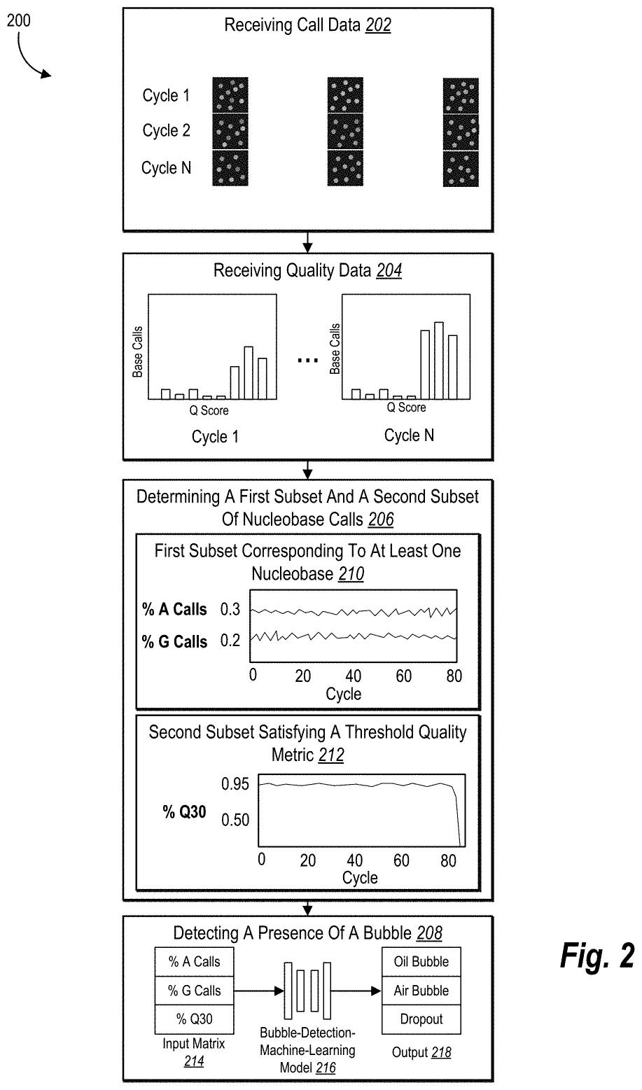

As indicated above, the bubble-detection system 106 can detect the presence of a bubble within a nucleotide-sample slide. For example, FIG. 2 illustrates the bubble-detection system 106 performing a series of acts 200 to detect a presence of a bubble within a nucleotide-sample slide in accordance with one or more embodiments. As part of the series of acts 200, the bubble-detection system 106 performs an act 202 of receiving call data, an act 204 of receiving quality data, an act 206 of determining a first subset and a second subset of nucleobase calls, and an act 208 of detecting a presence of a bubble.

As shown in FIG. 2, the series of acts 200 includes the act 202 of receiving call data. In particular, when performing the act 202, the bubble-detection system 106 receives call data comprising or indicating nucleobase calls for cycles of sequencing a nucleic-acid polymer. In some cases, the bubble-detection system 106 accesses call data from a sequencing device (e.g., imaging data from the sequencing device 114) indicating nucleobase calls for each sequencing cycle. For example, and as illustrated in FIG. 2, the bubble-detection system 106 receives image data for each cycle comprising intensity values indicating adenine (A) calls, thymine (T) calls, cytosine (C) calls, or guanine (G) calls for each sequencing cycle and section of a nucleotide-sample slide. In some embodiments, the call data also indicates the total number or percentage of particular nucleobases called within a particular cycle. Although FIG. 2 depicts the call data as image data with colors indicating intensity values, the bubble-detection system 106 can receive call data in any suitable format, such as call data as part of binary base call (BCL) sequence files or InterOp metric files.

In addition or in the alternative to receiving image data when performing the act 202, in certain implementations, the bubble-detection system 106 receives call data comprising individual nucleobase calls across cycles of sequencing a nucleic-acid polymer. For instance, in some cases, the call data includes explicit data or text indicators for an A, T, C, or G call for a particular cycle and section of a nucleotide-sample slide. As above, the call data may also include the total number or percentage of particular nucleobases called within a particular cycle.

As further illustrated in FIG. 2, the series of acts 200 includes the bubble-detection system 106 performing the act 204 of receiving quality data. As indicated above, quality data comprises quality metrics that estimate errors in the nucleobase calls for the cycles. In particular, the bubble-detection system 106 receives quality data from a sequencing device indicating a probability of erroneous nucleobase calls for each cycle. As illustrated in FIG. 2, for instance, the quality data comprises quality metrics corresponding to the total number of bases called for each cycle. Although FIG. 2 depicts the quality data as a distribution of total base calls associated with particular quality metrics, the bubble-detection system 106 can receive quality data in any suitable format, such as quality metrics within BCL files or InterOp metric files. In one or more embodiments, the quality data comprises quality metrics as described in additional detail below.

As further indicated above, in some embodiments, the quality metrics comprise quality scores associated with probabilities of an incorrect nucleobase call or base-call accuracies. For instance, in one or more embodiments, the quality metrics comprise Phred quality scores based on a Phred algorithm or a modified Phred algorithm developed by Illumina, Inc. In some embodiments, the bubble-detection system 106 determines or uses Phred scores as quality metrics as described by Method and System for Determining the Accuracy of DNA Base Identifications, U.S. Pat. No. 8,392,126 (filed Sep. 23, 2009), the contents of which are hereby incorporated by reference in their entirety. A Phred quality score of Q10 is equivalent to the probability of an incorrect nucleobase call 1 in 10 times, meaning that every 10 nucleobase sequencing read likely contains an error. The following table includes additional Phred quality scores and their equivalent probabilities of incorrect nucleobase calls and nucleobase call accuracy.

| Phred | Probability of | Nucleobase |
|---|---|---|
| quality | incorrect | call |
| score | nucleobase call | accuracy |
| Q10 | 1 in 10 | 90% |
| Q20 | 1 in 100 | 99% |
| Q30 | 1 in 1,000 | 99.9% |
| Q40 | 1 in 10,000 | 99.99% |
| Q50 | 1 in 100,000 | 99.999% |

Additional detail regarding Phred quality scores is provided by Ewing B, Green P. Base-calling of Automated Sequencer Traces Using Phred. II. Error Probabilities. Genome Res. 1998 March; 8(3):186-194. PMID: 9521922, the entirety of which is incorporated herein by reference.

As further illustrated in FIG. 2, the series of acts 200 includes the act 206 of determining a first subset and a second subset of nucleobase calls. In particular, when performing the act 206, the bubble-detection system 106 determines a first subset of the nucleobase calls corresponding to at least one nucleobase and a second subset of the nucleobase calls satisfying a threshold quality metric for the quality metrics. In some embodiments, the first subset and the second subset comprise a proportion or percentage of all nucleobase calls for a given cycle and a particular section of a nucleotide-sample slide (e.g., a tile). The following paragraphs provide additional detail regarding the first subset and the second subset.

As illustrated by FIG. 2, the bubble-detection system 106 determines a first subset corresponding to at least one nucleobase 210. For instance, and as illustrated in FIG. 2, the bubble-detection system 106 determines a subset of adenine calls and a subset of guanine calls for each cycle. In one or more embodiments, the first subset comprises a percentage value indicating a portion of all nucleobase calls that correspond to a particular nucleobase. While FIG. 2 illustrates the bubble-detection system 106 determining the first subset corresponding to at least one nucleobase 210 by determining a percentage of adenine calls and a percentage of guanine calls, the bubble-detection system 106 can also determine a first subset comprising any combination of adenine calls, thymine calls, cytosine calls, and guanine calls.

As further illustrated in FIG. 2, the bubble-detection system 106 also determines the second subset satisfying a threshold quality metric 212. The bubble-detection system 106 identifies a threshold quality metric and determines a subset of nucleobase calls that satisfy the threshold quality metric. In some implementations, the bubble-detection system 106 determines a threshold quality metric comprising a percentage or proportion of nucleobase calls that meet or exceed a benchmark threshold quality metric. To illustrate, in one or more embodiments, the bubble-detection system 106 determines that the threshold quality metric equals a Phred quality score of Q30. The bubble-detection system 106 determines, for each cycle, a percentage (or other subset) of nucleobase calls that meets or exceeds the Q30 quality metric.

After performing the act 206 of determining the first subset and the second subset of nucleobase calls, the bubble-detection system 106 performs the act 208 of detecting a presence of a bubble. In particular, when performing the act 208, the bubble-detection system 106 detects a presence of a bubble within the nucleotide-sample slide by utilizing a bubble-detection-machine-learning model based on the first subset of the nucleobase calls and the second subset of the nucleobase calls. As illustrated in FIG. 2, for example, the bubble-detection system 106 utilizes a bubble-detection-machine-learning model 216 to analyze an input matrix 214 and generate an output 218.

In addition to the series of acts 200, in some cases, the bubble-detection system 106 further provides an alert to a computing device indicating the presence of the bubble. In particular, the bubble-detection system 106 provides a notice or alert for display via the computing device associated with a user. Additionally, or alternatively, the bubble-detection system 106 provides the alert to the sequencing device. In any case, the bubble-detection system 106 can include, within the alert, an error classification that indicates the type of bubble or error. Furthermore, the alert can include additional information including the section of the nucleotide-sample slide and/or the sequencing cycle at which the bubble occurred.

Furthermore, in some implementations, the bubble-detection system 106 determines one or more corrective actions based on detecting the presence of a bubble. To illustrate, in some implementations, the bubble-detection system 106 reduces a quality metric for a particular read in a cycle, a particular cycle, or a particular section of a nucleotide-sample slide based on detecting the presence of a bubble. In some cases, for instance, the bubble-detection system 106 can identify the nucleobase calls in a cycle for which to reduce quality metrics by identifying a Unique Molecular Identifier (UMI) for the corresponding read. Additionally, or alternatively, the bubble-detection system 106 can, based on identifying a particular read in a cycle, a particular cycle, or a particular section of a nucleotide-sample slide impacted by a bubble, excise the affected call from the call data. In some cases, based on determining that the persistence of a bubble, the bubble-detection system 106 can include, within an alert, a suggested action to resolve a bubble. For example, based on determining that the number of detected oil bubbles meets a threshold value, the bubble-detection system 106 provides an alert including a suggested action to check parts of the sequencing device for oil leaks or to reload the nucleotide-sample slide.

As mentioned previously, in some embodiments, the bubble-detection system 106 identifies a particular section of a nucleotide-sample slide that is affected by a bubble. In one example, a section of a nucleotide-sample slide comprises a tile of a flow cell. Thus, in one or more embodiments, the bubble-detection system 106 performs the series of acts 200 for a particular section of a nucleotide-sample slide. Accordingly, in certain implementations, the bubble-detection system 106 receives call data and quality data across cycles for a single section of a nucleotide-sample slide. Thus, the bubble-detection system 106 can identify a particular section of a nucleotide-sample slide affected by a bubble.

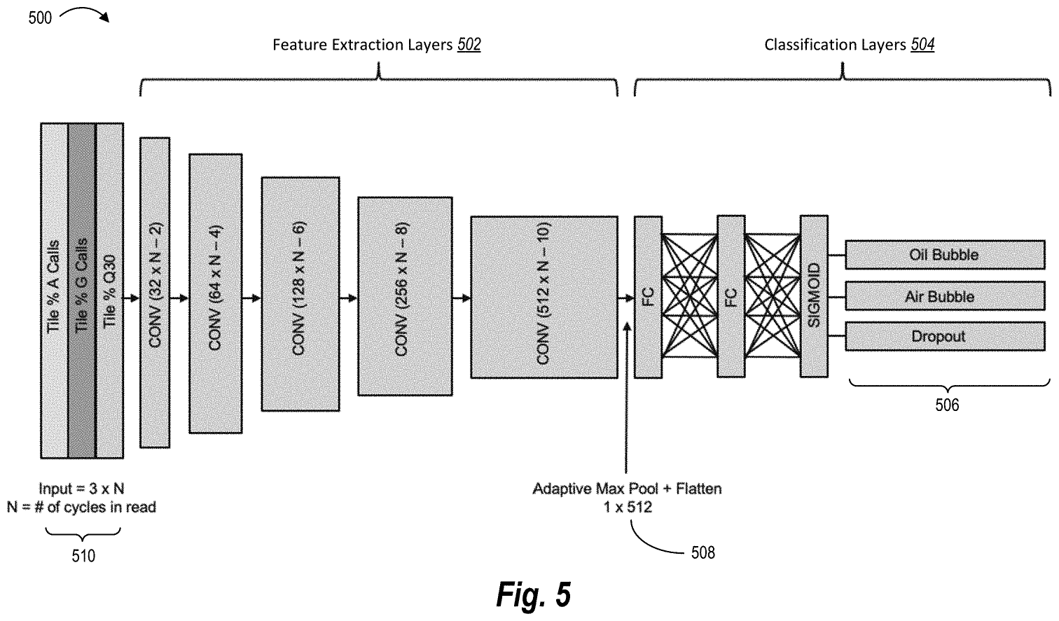

As further illustrated in FIG. 2, the bubble-detection system 106 utilizes the input matrix 214 as input into the bubble-detection-machine-learning model 216. In one or more embodiments, the input matrix 214 comprises data for the first subset of nucleobase calls corresponding to at least one nucleobase (e.g., the subset of adenine calls and the subset of guanine calls) and the second subset of the nucleobase calls satisfying the threshold quality metric. As described below with respect to FIG. 5, the input matrix 214 can vary in size based on the number of sequencing cycles.

As further indicated by FIG. 2, the bubble-detection system 106 implements the bubble-detection-machine-learning model 216. The bubble-detection-machine-learning model 216 extracts features from the input matrix 214 to identify the presence of a bubble within the nucleotide-sample slide. The bubble-detection-machine-learning model 216 can comprise various types of machine learning models. In some embodiments, the bubble-detection-machine-learning model 216 comprises a neural network, such as a CNN, or different types of machine learning models, such as an SVM or an Adaptive Boosting machine learning model. FIG. 5 and the corresponding discussion further describe an example CNN in accordance with one or more embodiments.

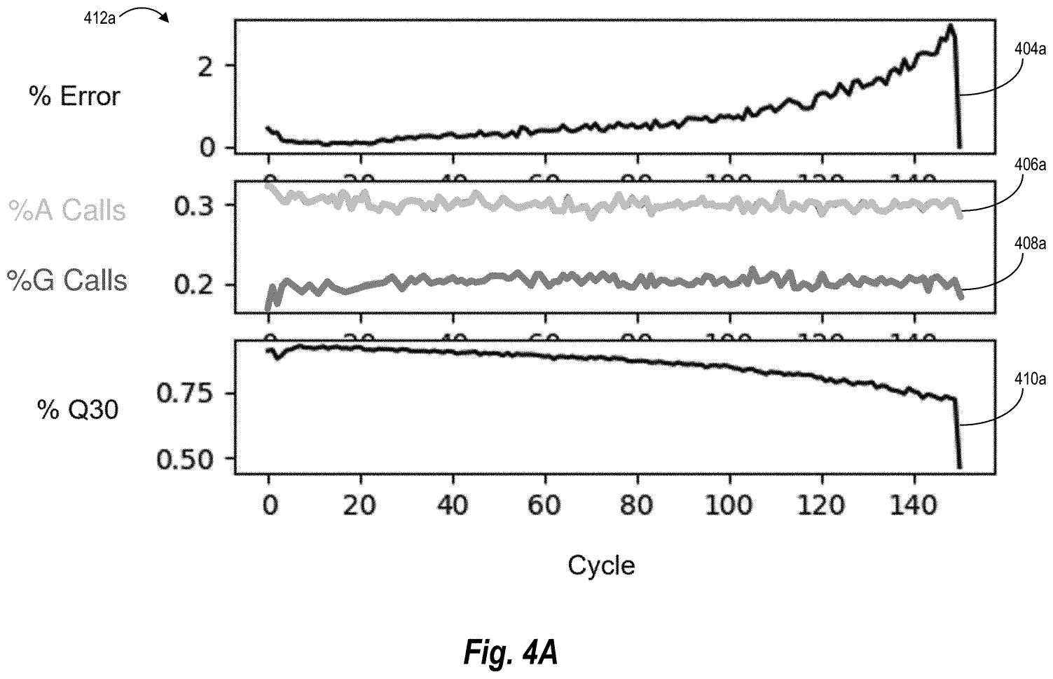

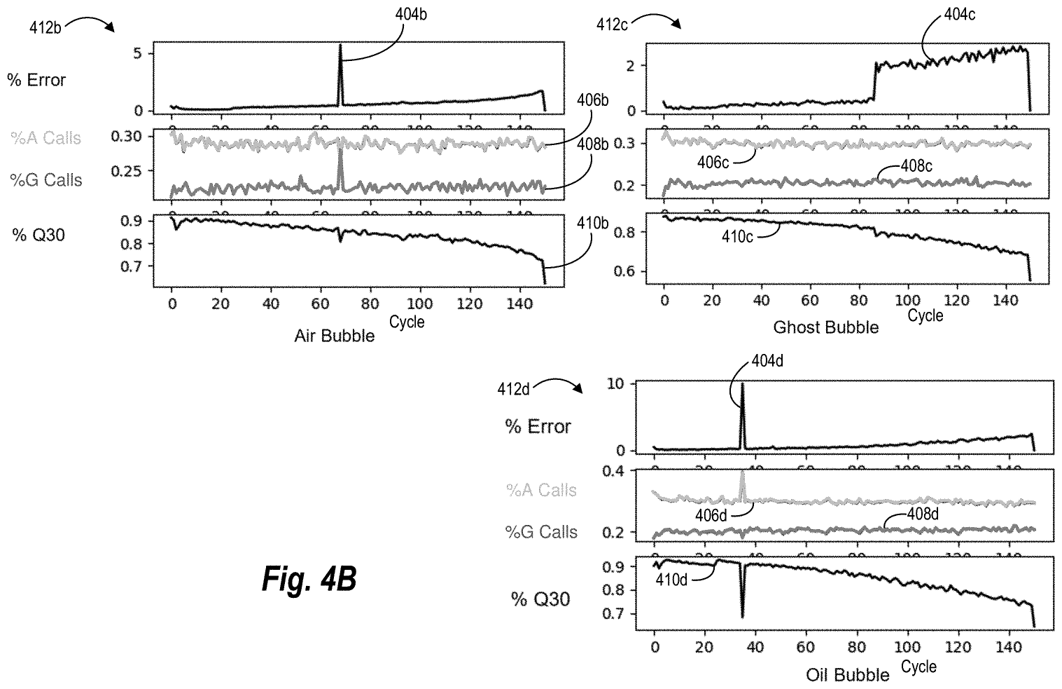

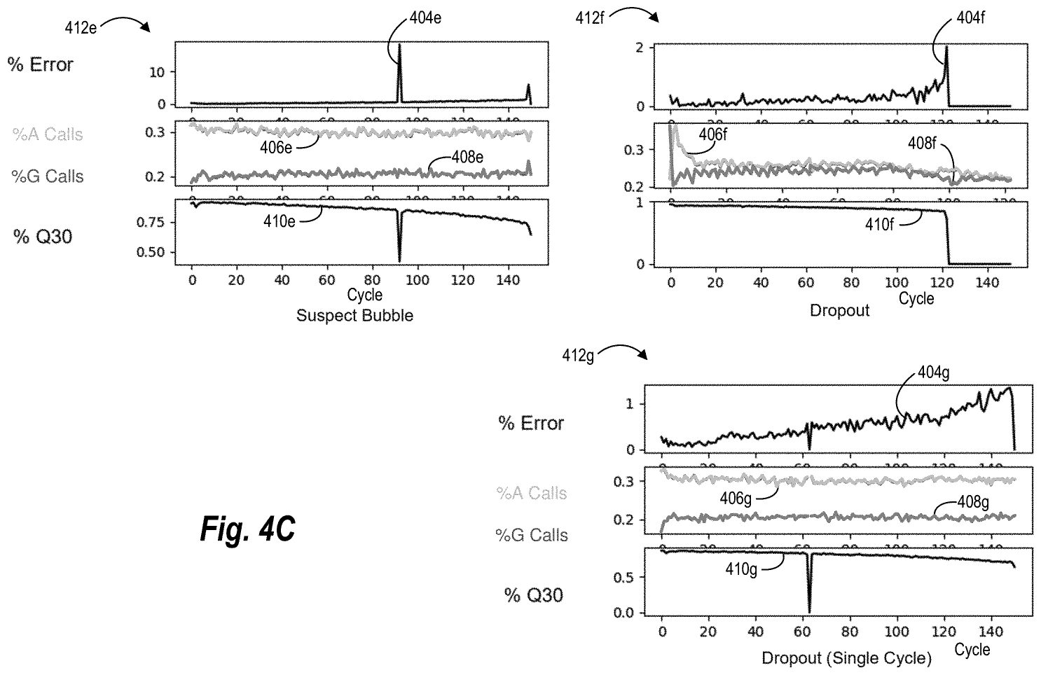

After passing the input matrix 214 through the bubble-detection-machine-learning model 216, the bubble-detection system 106 generates the output 218 utilizing the bubble-detection-machine-learning model 216. In some embodiments, the output 218 comprises (i) an indication of a bubble within the nucleotide-sample slide and (ii) an error classification. As illustrated in FIG. 2, for instance, the output 218 includes potential error classifications including an oil bubble, an air bubble, and dropout. In additional embodiments, the output 218 includes the additional error classifications of ghost bubbles. FIGS. 4A-4C and the corresponding paragraphs further describe the error classifications generated by the bubble-detection system 106 according to one or more embodiments.

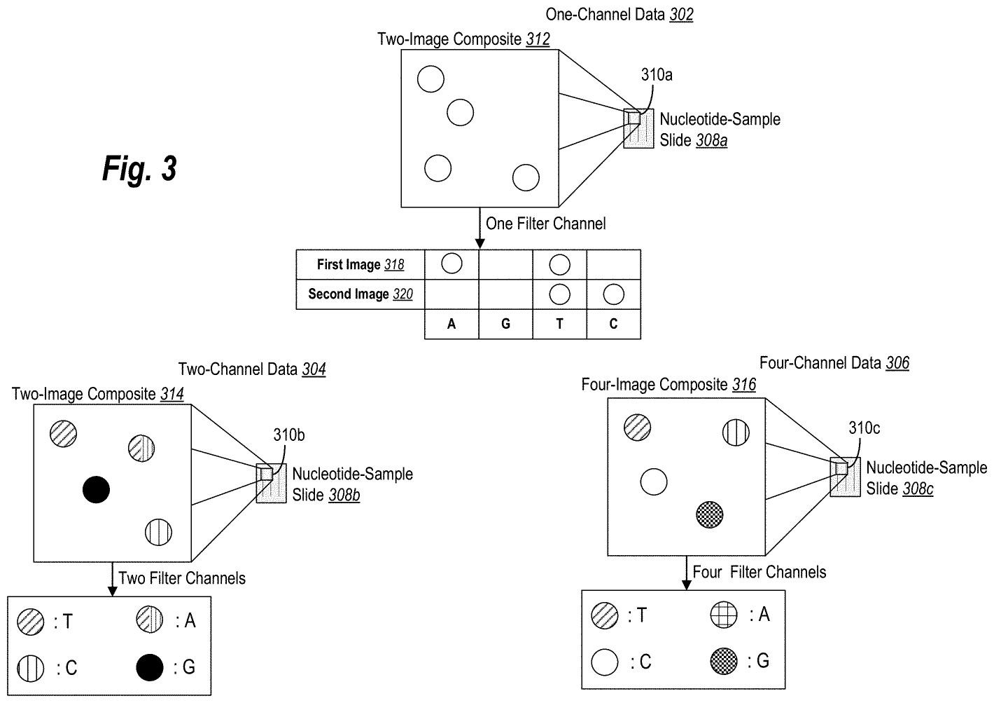

FIG. 2 provides a general overview of the bubble-detection system 106 determining the presence of a bubble within a nucleotide-sample slide in accordance with one or more embodiments. As mentioned, the bubble-detection system 106 can flexibly determine the presence of a bubble based on various types of call data. FIG. 3 illustrates different types of call data that the bubble-detection system 106 can utilize in determining the presence of a bubble within a nucleotide-sample slide. Generally, FIG. 3 illustrates one-channel data 302, two-channel data 304, and four-channel data 306 obtained as part of SBS cycles. The following paragraphs further describe each of these types of data.

As illustrated in FIG. 3, in some embodiments, the call data can comprise image data in the form of the one-channel data 302. In some embodiments, and as illustrated in FIG. 3, the one-channel data comprises a two-image composite 312 of a section 310a of a nucleotide-sample slide 308a for a given cycle of sequencing the nucleic-acid polymer. In certain embodiments, the two-image composite 312 comprises a combination of two images, each captured using the same detection channel, same dye, or same fluorescent label captured at different times. Unlike four-channel SBS chemistry where sequencers use a different fluorescent dye or label for each nucleobase, one-channel SBS chemistry uses one fluorescent dye, two chemistry steps, and two imaging steps (that produce two images) per sequencing cycle. In one-channel chemistry, for example, adenine has a removable label and is labeled in a first image 318 only. Cytosine has a linker group that can bind a label and is labeled in a second image 320 only. Thymine has a permanent fluorescent label and is therefore labeled in both the first image 318 and the second image 320. Guanine is not labeled and therefore does not fluoresce in either image. The bubble-detection system 106 determines nucleobase calls based on analyzing the different emission patterns for each base across the two images.

In one or more embodiments, the bubble-detection system 106 obtains one-channel data based on intensity information. In such embodiments, instead of capturing two images, the sequencing system 104 captures a single image and associates different intensities values with the different nucleobases. In particular three or more of the nucleobases bind the one fluorescent dye or label with different intensities. The bubble-detection system 106 can associate intensity ranges with particular nucleobases or the lack of a dye or label with a particular nucleobase. Accordingly, the bubble-detection system 106 determines the nucleobase calls based on intensity data using a single channel.

As further illustrated in FIG. 3, in certain cases, the bubble-detection system 106 receives call data in the form of the two-channel data 304. In particular, the two-channel data 304 comprises a two-image composite 314 of a section 310b of nucleotide-sample slide 308b. In particular, the two-image composite 314 comprises two images, each captured using a detection channel that specific to two different dyes or different fluorescent labels. Two-channel SBS simplifies nucleotide detection relative to four-channel SBS chemistry by using two fluorescent dyes and the two-image composite 314 to determine all four nucleobase calls. For example, in one embodiment, a camera of a sequencing device captures images using red and green filter bands. Thymine nucleobases are labeled with a green fluorophore, cytosines are labeled with a red fluorophore, and adenines are labeled with both red and green flourophores. Guanines are permanently dark. The bubble-detection system 106 determines nucleobase calls by using two filter channels to process the two-image composite 314 and to determine which nucleobases are incorporated within each cluster within the section 310b of the nucleotide-sample slide 308b.

As further noted above, in some implementations, the bubble-detection system 106 receives call data in the form of four-channel data 306. In particular, the four-channel data 306 comprises a four-image composite 316 of a section 310c of a nucleotide-sample slide 308c. In particular, the four-image composite 316 comprises four images, each captured using a detection channel that specific to one of the four different dyes or fluorescent labels. The four-channel SBS cycle begins with a chemistry step where all four different labeled bases are added to the nucleotide-sample slide. The imaging cycle begins and includes the capture of the four-image composite 316 using four different filter channels or wavelength bands. The bubble-detection system 106 processes the four-image composite 316 to determine which nucleobases are incorporated at each cluster position across the nucleotide-sample slide.

The bubble-detection system 106 determines subsets of nucleobase calls based on the call data. In particular, the bubble-detection system 106 stores, processes, and analyzes the one-channel data 302, the two-channel data 304, and/or the four-channel data 306 to determine base calls for each sequencing cycle. More specifically, the bubble-detection system 106 identifies nucleobases by analysis of different emission patterns for each nucleobase across the captured images. Upon completion of a sequencing cycle, the bubble-detection system 106 determines a total number of nucleobase calls. The bubble-detection system further determines subsets of individual nucleobase calls by comparing a number of a particular nucleobase call with the total number of nucleobase calls for a cycle. In one example, the bubble-detection system 106 determines 310 adenine calls out of 1000 total base calls for a given cycle. Based on this determination, the bubble-detection system 106 determines that the subset of adenine calls (% A calls) equals 0.31.

As mentioned previously, as part of detecting a presence of a bubble within a nucleotide-sample slide, in some embodiments, the bubble-detection system 106 utilizes the bubble-detection-machine-learning model to generate error classifications based on a subset of adenine calls, a subset of guanine calls, and a subset of nucleobase calls satisfying a threshold quality metric for the cycles of sequencing the nucleic-acid polymer. For instance, in certain embodiments, the bubble-detection system 106 generates error classifications identifying an error as caused by an air bubble, oil bubble, ghost bubble, or dropout. Each error classification corresponds with a different data signature for metrics from call data and quality data.

The bubble-detection system 106 can detect bubbles or classify such errors that correspond to various data signatures depicted in FIGS. 4A-4C. In accordance with one or more embodiments, FIGS. 4A, 4B, and 4C illustrate example charts that graph the progression of the input data depicted as data signatures over cycles within a sequencing run. In particular, FIG. 4A illustrates a data chart indicating the example data signature corresponding to a nucleotide-sample slide with no bubbles. FIG. 4B illustrates example data signatures corresponding to an air bubble, a ghost bubble, and an oil bubble in accordance with one or more embodiments. FIG. 4C illustrates example data signatures corresponding to a suspect bubble, dropout, and dropout occurring within a single cycle in accordance with one or more embodiments. While FIGS. 4A-4C depict charts for data input into a bubble-detection-machine-learning model—including a subset of adenine calls, a subset of guanine calls, and a subset of nucleobase calls satisfying a threshold value metric—the bubble-detection system 106 does not input the charts themselves into such a model.

As an overview, the charts in FIGS. 4A-4C share some common features. For example, FIGS. 4A-4C illustrate example charts 412a-412g with data signatures corresponding to various error classifications. The metrics graphed by the charts 412a-412g illustrated include error percentage 404a-404g, adenine-calls percentages 406a-406g, guanine-calls percentages 408a-408g, and Q30-satisfying percentages 410a-410g. More specifically, the charts 412a-412g indicate the progression of the metrics over the sequencing cycles within a sequencing run. The error percentage 404a-404g indicate the percentage of error predicted for nucleobase calls in each cycle. The adenine-calls percentages 406a-406g indicate a percentage (or subset) of all nucleobase calls in each cycle that comprise adenine calls. Similarly, the guanine-calls percentages 408a-408g indicate a percentage (or subset) of all nucleobase calls in each cycle that comprise guanine calls. The Q30-satisfying percentages 410a-410g indicate a percentage of nucleobase calls in each cycle that satisfy a Q30 threshold quality metric. In one or more other embodiments, the bubble-detection system 106 extracts features from other metrics to identify and classify errors.

As mentioned, FIG. 4A illustrates a chart 412a associated with no bubbles. In particular, the chart 412a displays data signatures for a nucleotide-sample slide containing no bubbles. Generally, no bubbles correspond with data signatures with relatively steady metrics. For instance, the error percentage 404a, the adenine-calls percentage 406a, the guanine-calls percentage 408a, and the Q30-satisfying percentage 410a remain relatively stable over the sequencing cycles. The chart 412a provides a baseline for comparing charts corresponding to different errors. Based on data corresponding to the chart 412a, the bubble-detection system 106 would not detect a presence of a bubble.

By contrast, FIG. 4B illustrates a chart 412b with data signatures indicating an air bubble, a chart 412c with data signatures indicating a ghost bubble, and a chart 412d with data signatures indicating an oil bubble. For instance, the chart 412b includes metrics in data signatures reflecting nucleobase calls for a nucleotide-sample slide containing an air bubble. Generally, air bubbles result from air entering fluidic lines and channels within the nucleotide-sample slide. Air bubbles negatively impact data quality of sequencing reads when they occur and are captured during the imaging stage of a sequencing cycle. For instance, during the imaging stage, air bubbles can obscure parts of images or reduce chemistry efficiency. More specifically, air bubbles can enter the nucleotide-sample slide from gaskets of the nucleotide-sample slide and laminate outgassing during imaging.

As indicated by the chart 412b, an air bubble causes a spike in both the error percentage 404b and the guanine-calls percentage 408b—while also causing a dip in the adenine-calls percentage 406b and the Q30-satisfying percentage 410b. As further illustrated in FIG. 4B, the sequencing device captured the air bubble between the 60th and 80th sequencing cycles. Based on data corresponding to the data signatures shown in the chart 412b, the bubble-detection system 106 would detect a presence of a bubble and classify the bubble as an air bubble.

As further shown in FIG. 4B, the chart 412c graphs metrics for a nucleotide-sample slide containing a ghost bubble. A ghost bubble refers to an air or an oil bubble that occurs outside of the imaging stage. For example, in contrast to air bubbles and oil bubbles that occur when a camera of the sequencing device takes a picture of the nucleotide-sample slide, ghost bubbles impact quality data by affecting chemistry steps leading up to (and following) the imaging stage. For example, ghost bubbles may occur during incorporation when primers and nucleotides are washed onto the nucleotide-sample slide or during deblocking when fluorescent terminal blocking groups are removed.

As illustrated in the chart 412c, a ghost bubble occurring sometime after the 80th sequencing cycle causes the error percentage 404c to rapidly increase and stay elevated for the remaining sequencing cycles. Additionally, the Q30-satisfying percentage 410c mirrors the error percentage 404c and dips at the same sequencing cycle. As further illustrated in the chart 412c, the adenine-calls percentage 406c and the guanine-calls percentage 408c remain relatively similar to the control. Based on data corresponding to the data signatures shown in the chart 412c, the bubble-detection system 106 would detect a presence of a bubble and classify the bubble as a ghost bubble.

As also depicted in FIG. 4B, the chart 412d graphs metrics for a nucleotide-sample slide containing an oil bubble. Generally, oil bubbles occur when oil from parts of the sequencing device enters the nucleotide-sample slide. Similar to air bubbles, oil bubbles negatively impact data quality by impacting the images captured during the imaging stage of a sequencing cycle. More particularly, oil bubbles absorb dyes or labels and fluoresce, causing the sequencing device to capture excessive fluorescence. For example, and as illustrated by the chart 412d, an oil bubble captured between the 20th and 40th sequencing cycles cause a sharp peak in the error percentage 404d and the adenine-calls percentage 406d. The chart 412d also graphs a smaller dip in the guanine-calls percentage 408d with a more pronounced dip in the Q30-satisfying percentage 410d. Based on data corresponding to the data signatures shown in the chart 412d, the bubble-detection system 106 would detect a presence of a bubble and classify the bubble as an oil bubble.

As indicated above, FIG. 4C illustrates example charts corresponding to additional error classifications. In particular, FIG. 4C illustrates a chart 412e corresponding to a suspect bubble, a chart 412f corresponding to dropout, and a chart 412g corresponding to dropout within a single cycle.

As shown in FIG. 4C, for instance, the chart 412e graphs metrics for a nucleotide-sample slide with a suspect bubble. Generally, a suspect bubble can indicate no bubble, one of the previously mentioned bubbles (e.g., air bubble, ghost bubble, oil bubble), or another type of error. In particular, while certain bubble classifications (e.g., air bubble, ghost bubble, and oil bubble) are linked with distinct data signatures, such data signatures may also include some variance. Additionally, other errors, beside bubbles, may affect the quality of data. Thus, in some embodiments, the bubble-detection system 106 generates a classification of “no bubble” based on a subset of nucleobase calls corresponding to the data signatures in the chart 412e. Alternatively, in certain implementations, the bubble-detection system 106 generates a classification of “unknown bubble type” or “unknown error type” based on a subset of nucleobase calls corresponding to the data signatures in the chart 412e. In one or more embodiments, the suspect bubble classification corresponds to data signatures that vary slightly from the typical data signature of a particular bubble classification or a no-bubble data signature (e.g., as illustrated in FIG. 4A).

To illustrate, the chart 412e shows a peak in the error percentage 404e and a corresponding dip in the Q30-satisfying percentage 410e. But the adenine-calls percentage 406e and the guanine-calls percentage 408e of the chart 412e remain relatively unaffected. In one or more embodiments, the bubble-detection system 106 determines a classification of a suspect bubble based on features of an input matrix that are similar to but beyond a threshold difference with features of air, oil, or ghost bubbles. Based on data corresponding to the data signatures shown in the chart 412e, the bubble-detection system 106 would detect a presence of a bubble, but not classify the bubble.

FIG. 4C further illustrates the charts 412f and 412g corresponding to nucleotide-sample slides with dropout. Generally, dropout refers to when a camera captures no or a limited amount of image data for a section (e.g., a tile within a flow cell) or a cluster within a section of a nucleotide-sample slide. Such dropout differs from and does not refer to image data with a dark signal or intensity values indicating a nucleotide lacking a particular fluorescent label or a nucleotide with a label not irradiated by a particular wavelength of light. Dropout can occur in various stages of the sequencing cycle. As shown by the chart 412f, dropout can occur during cluster or section registration stages of SBS sequencing. Additionally, and as shown by the chart 412g, dropout can occur in a single cycle.

As mentioned, the chart 412f illustrates the effects of dropout that occurs during cluster or section registration. Generally, clusters refer to a group of nucleic-acid segments or cloned segments from a sample. In particular, a cluster represents thousands of copies of the same DNA or RNA segment. For example, in one or more embodiment, a cluster is immobilized in a section of a nucleotide-sample slide. In some embodiments, clusters may be evenly spaced using a patterned nucleotide-sample slide.

During cluster and section registration, the sequencing system 104 records locations of clusters and sections for imaging. In some embodiments, the sequencing system 104 also records intensity values during cluster and section registration. Generally, dropout that occurs during cluster registration causes the sequencing system 104 to fail to register a particular cluster for the duration of the sequencing cycles. As illustrated by the chart 412f, dropout that occurs during section or cluster registration results in longer lasting effects. In particular, the error percentage 404f indicates a sharp increase around the 120th sequencing cycle and the Q30-satisfying percentage 410f indicates a corresponding drop. Based on data corresponding to the data signatures shown in the chart 412f, the bubble-detection system 106 would detect a dropout event during registration.

Dropout occurring during cluster and section registration can have various causes. For instance, dropout during cluster registration may indicate the presence of a bubble that covers the entire section of a nucleotide-sample slide. Additionally, dropout during cluster registration may indicate other types of irregularities. For example, dropout may indicate errors in software or hardware function. In one example, dropout indicates Direct Memory Access (DMA) transfer fails between the sequencing device and the user client device or the server device(s). Additionally, dropout may signal a hardware failure in sensors or cameras which results in the excision of data relating to the particular nucleotide-sample slide section or cluster. For instance, sensors within the sequencing device may be out of focus.

As further illustrated by the chart 412g of FIG. 4C, the bubble-detection system 106 can detect dropout that occurs during a sequencing cycle. In particular, during a given cycle, the sequencing device may erroneously omit data for a cluster or section of a nucleotide-sample slide. For example, the sequencing device may suffer a mechanical error that causes sensors to drop entire clusters or sections of the nucleotide-sample slide during the cycle. In another example, the sequencing device suffers a Real Time Analysis (RTA) error that causes dropout during the sequencing run. As illustrated by the chart 412g, dropout in a single sequencing cycle may manifest in a pronounced dip in the Q30-satisfying percentage 410g and a smaller corresponding dip in the error percentage 404g. Furthermore, both the adenine-calls percentage 406g and the guanine-calls percentage 408g have data gaps corresponding to the cycle affected by the dropout. Based on data corresponding to the data signatures shown in the chart 412f, the bubble-detection system 106 would detect a dropout event during a single cycle.

FIGS. 4B-4C illustrate example charts displaying data signatures of various error classifications. In some embodiments, the bubble-detection system 106 utilizes a bubble-detection-machine-learning model to extract features from an input matrix and determine the presence of a bubble and a corresponding classification for the bubble. As mentioned previously, the bubble-detection-machine-learning model can comprise a neural network. FIG. 5 illustrates an example configuration of a bubble-detection-neural network in accordance with one or more embodiments. In particular, FIG. 5 illustrates a bubble-detection-neural network 500 comprising feature extraction layers 502, classification layers 504, and an adaptive max pooling layer 508. As illustrated, the bubble-detection-neural network 500 comprises a trained neural network that the bubble-detection system 106 applies to an input matrix 510. The bubble-detection system 106 further generates output classifications 506 by utilizing the bubble-detection-neural network 500.

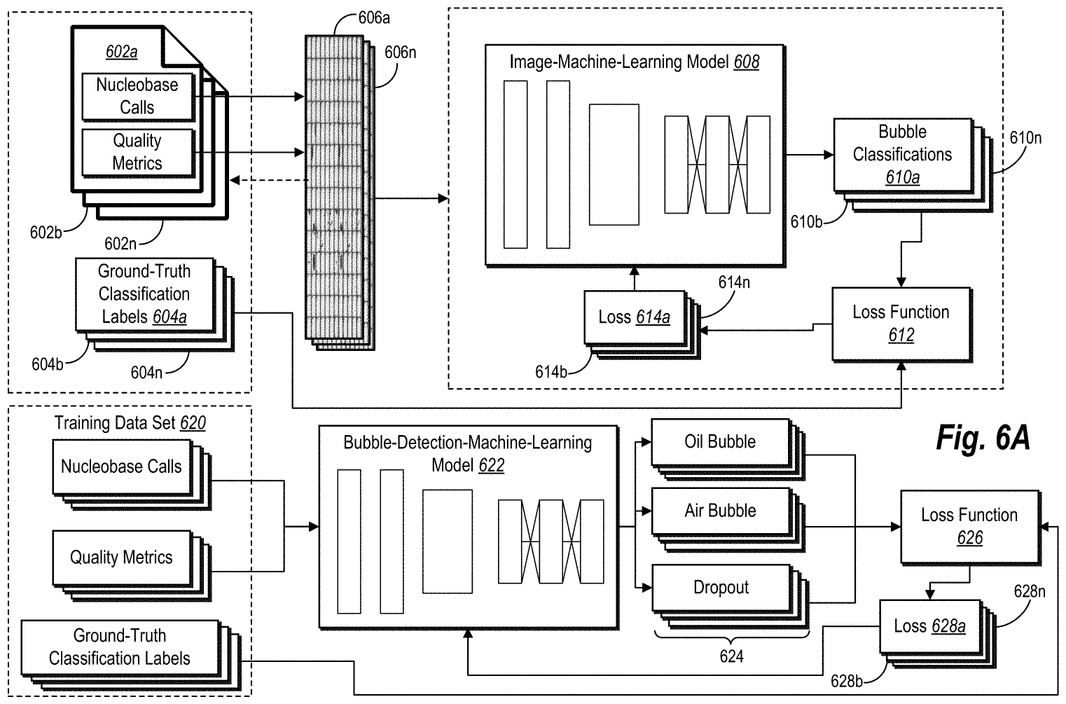

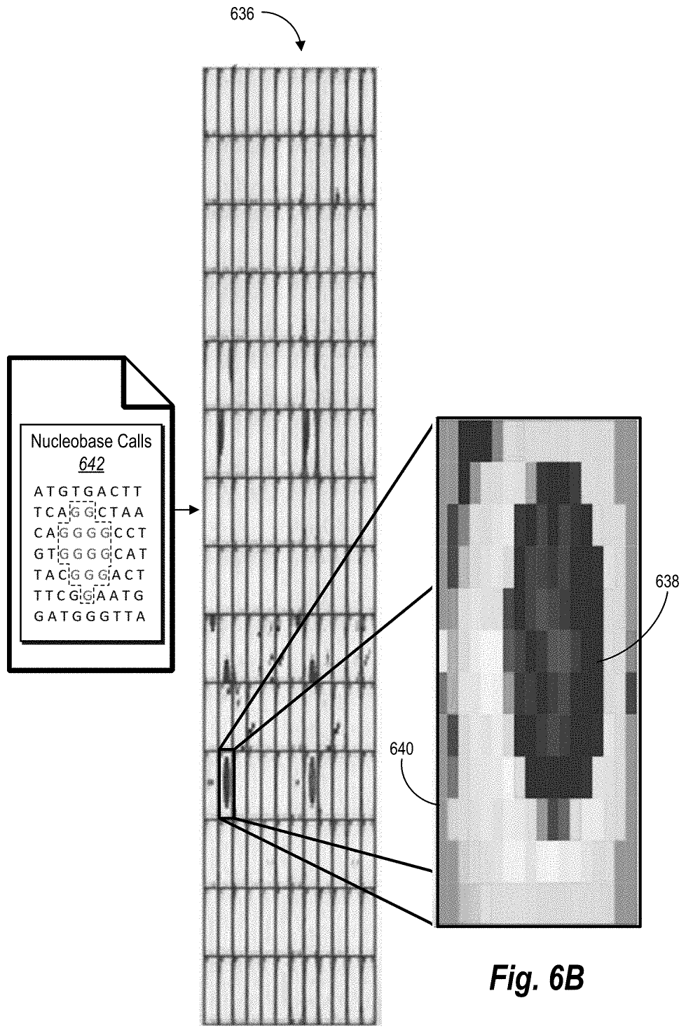

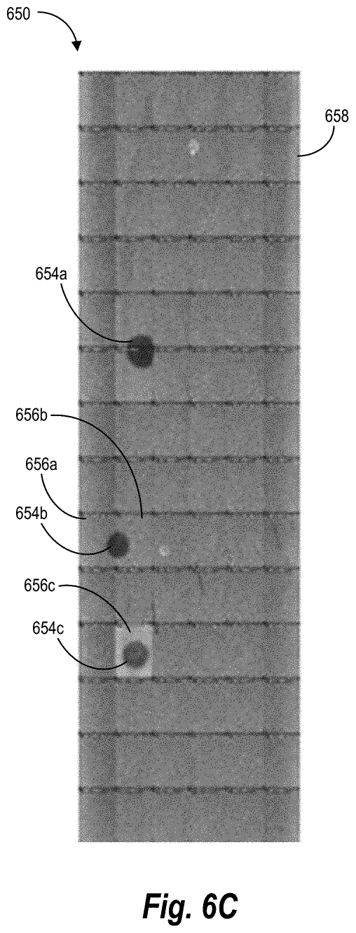

As shown in FIG. 5, the bubble-detection-neural network 500 comprises a trained neural network. In particular, in one or more embodiments, the bubble-detection system 106 trains the bubble-detection-neural network 500 utilizing a training data set. In one embodiment, the bubble-detection system 106 accesses a training data set comprising ground truth classifications for training input matrices. FIG. 6A and the corresponding discussion provides additional description regarding how the bubble-detection system 106 trains the bubble-detection-neural network 500 in accordance with one or more embodiments.

As further illustrated in FIG. 5, the bubble-detection system 106 applies the bubble-detection-neural network 500 to the input matrix 510 after training. As illustrated in FIG. 5, for each section of the nucleotide-sample slide (e.g., a tile of a flow cell), the input matrix 510 comprises three one-dimensional input channels of length N, where N equals the number of SBS cycles in the run. In some embodiments, the three one-dimensional input channels comprise a subset of adenine calls, a subset of guanine calls, and a subset of the nucleobase calls satisfying the threshold quality metric (e.g., % Q30). The size of the input matrix 510 is variable and can therefore accommodate a wide range of sequencing run lengths.

In addition to training a machine-learning model to detect and classify bubbles, in certain implementations, the bubble-detection system 106 trains such a model to discriminate between bubbles introduced during specific sequencing chemistry steps or stages. Bubbles occurring at different SBS or Sanger chemistry steps or stages can result in unique data signatures. By using training data corresponding to such unique data signatures specific to the chemistry step or stage at which a bubble enters or interferes with a nucleotide-sample slide, for instance, the bubble-detection system 106 can train a bubble-detection-machine-learning model to detect and discriminate between bubbles introduced during specific SBS chemistry steps or stages. In some embodiments, for example, the bubble-detection system 106 differentiates between bubbles introduced during sequencing steps (e.g., incorporation or deblock) or during imaging steps (e.g., scan mix of reagents in a flow cell).

As indicated above and shown in FIG. 5, in some embodiments, the bubble-detection-neural network 500 comprises a lightweight CNN. The bubble-detection-neural network 500 can comprise a CNN having lower network layers (e.g., convolutional and deconvolutional layers) and higher neural network layers (e.g., fully-connected layers). In alternative embodiments, the bubble-detection-neural network 500 employs a different neural network architecture. Furthermore, in some implementations, the bubble-detection-neural network 500 does not use downsampling methods, such as the implementation of max pooling layers to compress dimensions after convolution operations. In such implementations, the bubble-detection system 106 excludes max pooling layers to maintain representation size, especially for short sequencing runs (e.g., N=36).

As further illustrated in FIG. 5, the bubble-detection-neural network 500 includes the adaptive max pooling layer 508. In some implementations, the adaptive max pooling layer 508 is located between the feature extraction layers 502 and the classification layers 504 of the bubble-detection-neural network 500. By implementing the adaptive max pooling layer 508, the bubble-detection system 106 specifies the representation size and spatially collapses features for input into the classification layers 504. The implementation of the adaptive max pooling layer 508 improves the efficiency of the bubble-detection-neural network 500. In an alternative to the CNN shown in FIG. 5, in some cases, the bubble-detection-neural network 500 does not include the adaptive max pooling layer 508.

By using the adaptive max pooling layer 508, in some embodiments, the bubble-detection-neural network 500 becomes translation invariant. More specifically, translation invariant networks produce the same output for regardless of certain changes in the input. In one example, a translation invariant version of the bubble-detection-neural network 500 simply indicates the presence of and classification of a bubble within a nucleotide-sample slide section but does not indicate the particular cycle in which the bubble occurred. By removing or adjusting parameters of the adaptive max pooling layer 508, the bubble-detection system 106 can specify additional classifications to include in the output. For example, the bubble-detection-neural network 500 can generate, in addition to an error classification, an indication of the specific cycle in which a bubble occurred.

As indicated above, FIG. 5 illustrates the classification layers 504 as part of the bubble-detection-neural network 500. As illustrated here, the classification layers 504 comprise fully-connected neural networks that classify features extracted by the feature extraction layers 502. In one or more implementations, the classification layers 504 can generate multiclass outputs and indicate multiple error classifications for a single section of a nucleotide-sample slide. For example, the classification layers 504 can generate classifications of both oil bubble and air bubble for a single section.

As further illustrated in FIG. 5, the bubble-detection-neural network 500 includes the output classifications 506. In some embodiments, the bubble-detection-neural network 500 outputs a corresponding confidence or probability score. Based on determining that the confidence or probability score for a particular classification satisfies a confidence threshold, the bubble-detection system 106 determines the particular classification of either an oil bubble, air bubble, or a dropout for the input matrix 510. In other words, the bubble-detection system 106 detects a bubble or dropout event and classifies the same as either an oil bubble, air bubble, or a dropout based on a confidence score satisfying a particular threshold. While FIG. 5 illustrates oil bubble, air bubble, and dropout classifications, the output classifications 506 can include any number of additional classifications. For example, the output classifications 506 can include a ghost bubble classification, a registration dropout classification, an imaging dropout classification, a suspect bubble classification, and other error classifications.

The bubble-detection-neural network 500 in FIG. 5 illustrates an example configuration of a CNN in accordance with one or more implementations. In other embodiments, the bubble-detection system 106 utilizes machine learning models having various other configurations. Alternatively, the bubble-detection system 106 can utilize a neural network having a different configuration to identify a particular cycle affected by the bubble. For example, in certain implementations, the bubble-detection system 106 incorporates an attention layer in a CNN to generate classifications indicating a specific location (e.g., cluster, section) on the nucleotide-sample slide affected by a bubble. The bubble-detection system 106 can also implement other types of deep neural networks. For instance, the bubble-detection system 106 can implement a Long Short-Term Memory (LSTM) network or other type of recurrent neural network. Furthermore, in additional embodiments, the bubble-detection system 106 utilizes different types of machine learning models as the bubble-detection-neural network 500. In some examples, the bubble-detection system 106 utilizes an SVM or an Adaptive Boosting (AdaBoost) machine learning model.

In some embodiments, the bubble-detection system 106 uses nucleobase-call data corresponding to spatial images (or reconstructed spatial images) to detect the presence of a bubble within a section of a nucleotide-sample slide. For instance, and as mentioned previously, the bubble-detection system 106 can use spatial images of a section (e.g., tile) or sub-section (e.g., sub-tile) of a nucleotide-sample slide to train an image-machine-learning model to detect or classify bubbles. In some embodiments, for instance, the bubble-detection system 106 identifies ground-truth classification labels for nucleobase-call data (e.g., from BCL or BAM files) corresponding to spatial image data with correctly detected presence or absence or bubbles to train a bubble-detection-machine learning model (e.g., the bubble-detection-neural network 500).

As just suggested, FIGS. 6A-6C generally illustrate the bubble-detection system 106 training an image-machine-learning model and a bubble-detection-machine-learning model using nucleobase-call data corresponding to spatial images in accordance with one or more embodiments. In particular, FIG. 6A illustrates the bubble-detection system 106 training an image-machine-learning model using spatial images of nucleotide-sample-slide sections, generating ground-truth classification labels for such spatial images and corresponding nucleobase-call data, and utilizing the nucleobase-call data and ground-truth classification labels to further train a bubble-detection-machine-learning model. FIG. 6B illustrates an example spatial image generated by the bubble-detection system 106 in accordance with one or more embodiments. FIG. 6C illustrates an example sequencing run image depicting a portion of a nucleotide-sample slide in accordance with one or more embodiments.

As mentioned, in some implementations, the bubble-detection system 106 utilizes an image-machine-learning model 608 to detect or classify bubbles based on spatial images (or reconstructed spatial images) of sections or sub-sections of a nucleotide-sample slide. To illustrate, FIG. 6A depicts the bubble-detection system 106 training an image-machine-learning model 608 using spatial images 606a-606n and identifying nucleobase-call data 602a-602n and ground-truth classification labels 604a-604n corresponding to the spatial images 606a-606n. The bubble-detection system 106 subsequently uses the nucleobase-call data 602a-602n and the ground-truth classification labels 604a-604n to train a bubble-detection-machine-learning model 622. While FIG. 6A illustrates the bubble-detection system 106 training the image-machine-learning model 608, such training or use of the image-machine-learning model 608 is optional and represents one or more embodiments. Indeed, in some embodiments, the bubble-detection system 106 uses some or all of the nucleobase-call data 602a-602n and the ground-truth classification labels 604a-604n to train the bubble-detection-machine-learning model 622 without training or using the image-machine-learning model 608. FIG. 6A accordingly includes a dotted line around the image-machine-learning model 608 and corresponding outputs and determined losses to indicate such training and use is optional.

For simplicity, this disclosure describes an initial training iteration followed by a summary of subsequent training iterations depicted in FIG. 6A. By way of overview, in an initial training iteration depicted by FIG. 6A, the bubble-detection system 106 utilizes nucleobase-call data 602a to generate or reconstruct the spatial image 606a. The bubble-detection system 106 utilizes the spatial images 606a as an input for the image-machine-learning model 608 to subsequently generate bubble classifications 610a.

As just indicated and as illustrated in FIG. 6A, the bubble-detection system 106 utilizes the nucleobase-call data 602a-602n to generate the spatial images 606a-606n. In one or more embodiments, the nucleobase-call data 602a-602n comprises nucleobase calls and quality metrics corresponding to sections or sub-sections within a nucleotide-sample slide for a given sequencing cycle. In certain circumstances, the bubble-detection system 106 accesses the nucleobase-call data 602a-602n from BCL sequence files or BAM (*.bam) files. Some such nucleobase-call data may, for instance, include a pattern of nucleobase calls (e.g., a circular pattern of A calls or G calls) indicating a presence of a bubble within a tile or sub-tile of a nucleotide-sample slide.

As further illustrated in FIG. 6A, in one or more embodiments, the bubble-detection system 106 generates or reconstructs the spatial images 606a-606n based on the nucleobase-call data 602a-602n. Generally, the bubble-detection system 106 incorporates nucleobase calls into spatial patterns by generating spatial representations of the nucleobase calls from BCL or BAM files arranged according to the location of clusters on the nucleotide-sample slide. In one example, the bubble-detection system 106 color codes the spatial images 606a-606n by linking nucleobases with specific colors. For instance, the bubble-detection system 106 can associate A calls with yellow, G calls with blue, C calls as red, and T calls as green. The bubble-detection system 106 FIG. 6B illustrates an example spatial image in accordance with one or more embodiments.

In one or more embodiments, the bubble-detection system 106 reduces the size of the spatial images 606a-606n before inputting them into the image-machine-learning model 608. In at least one example, the bubble-detection system 106 down samples the spatial images 606a-606n. For instance, the bubble-detection system 106 processes the spatial images 606a-606n to remove high frequency information and retain low frequency information for the input. Thus, in some cases, the bubble-detection system 106 can apply the image-machine-learning model 608 to a low frequency version of the spatial images 606a-606n to improve efficiency.

After inputting the spatial images 606a as part of an initial training iteration, for instance, the bubble-detection system 106 executes the image-machine-learning model 608. As suggested above, the image-machine-learning model 608 may be a neural network, such as a CNN. In some cases, the image-machine-learning model 608 takes the form of a Dense Convolutional Network (DenseNet) or a Residual Neural Network (ResNet), to name a few examples.

As further illustrated in FIG. 6A, upon receiving the input data for an initial training iteration, the image-machine-learning model 608 determines bubble classifications 610a. Additionally, the image-machine-learning model 608 predicts the location of detected bubbles within sections or sub-sections of a nucleotide-sample slide based on spatial patterns within the input data. For instance, the image-machine-learning model 608 generates the bubble classifications 610a comprising labels indicating the presence and location of a bubble within a section of a nucleotide-sample slide. Generally, bubbles are associated with circular spatial patterns within the nucleobase-call data 602a or the spatial image 606a. Thus, in some embodiments, the bubble classifications 610a comprise a bubble classification together with a location of a bubble. For instance, the bubble classifications 610a can indicate a predicted section or sub-section of a nucleotide-sample slide that contains a bubble or a portion of a bubble. The bubble classifications 610a can likewise indicate a predicted section or sub-section of a nucleotide-sample slide that do not contain a bubble or a portion of a bubble.

As further illustrated in FIG. 6A, the bubble-detection system 106 uses a loss function 612 to compare the bubble classifications 610a with the ground-truth classification labels 604a. In some implementations, the ground-truth classification labels 604a comprise ground-truth bubble classifications and bubble locations corresponding to the nucleobase-call data 602a. For instance, the ground-truth classification labels 604a can indicate (i) a particular section or sub-section of a nucleotide-sample slide that contains a bubble or a portion of a bubble and (ii) a particular section or sub-section of a nucleotide-sample slide that contains no bubble or no portion of a bubble.

Depending on the form of the image-machine-learning model 608, the bubble-detection system 106 can use a variety of loss functions for the loss function 612. In certain embodiments, the bubble-detection system 106 uses a cross-entropy-loss function (e.g., for a CNN). For instance, the bubble-detection system 106 can use a pixelwise cross-entropy-loss function for a DenseNet or ResNet or some other suitable loss function (e.g., pixel-wise L1 or L2, feature-wise perceptual loss). Regardless of the form of the loss function 612, the bubble-detection system 106 determines the losses 614a-614n from the loss function 612 based on a comparison of the bubble classifications 610a with the ground-truth classification labels 604a. Indeed, in certain implementations, the losses 614a-614n may include separate losses for a particular section of a nucleotide-sample slide (e.g., tile or sub-tile).

Based on the determined losses 614a-614n from the loss function 612, the bubble-detection system 106 subsequently adjusts parameters of the image-machine-learning model 608. By adjusting the parameters, the bubble-detection system 106 increases the accuracy with which the image-machine-learning model 608 determines the presence and location of bubbles based on spatial images through multiple training iterations. Indeed, as further shown in FIG. 6A, the bubble-detection system 106 performs subsequent training iterations. As suggested by FIG. 6A, in some embodiments, the bubble-detection system 106 iteratively inputs spatial images 606b-606n into the image-machine-learning model 608 to generate bubble classifications 610b-610n, iteratively compares the bubble classifications 610b-610n to ground-truth classification labels 604b-604n to determine losses 614b-614n, and iteratively adjusts the parameters of the image-machine-learning model 608. In some cases, the bubble-detection system 106 performs training iterations until the parameters (e.g., value or weights) of the image-machine-learning model 608 do not change significantly across training iterations or otherwise satisfy a convergence criteria.

As suggested above, in some embodiments, the bubble-detection system 106 utilizes the image-machine-learning model 608 as part of identifying a training data set for a bubble-detection-machine-learning model. Additionally, or alternatively, in some embodiments, the bubble-detection system 106 utilizes the image-machine-learning model 608 as a bubble-detection-machine-learning model. In yet additional embodiments, the bubble-detection system 106 utilizes the image-machine-learning model 608 in addition to the bubble-detection-machine-learning model 622 to improve the accuracy of generated classifications. In one example, the bubble-detection system 106 utilizes the image-machine-learning model 608 to remove false positives generated by the bubble-detection-machine-learning model 622.

As just mentioned, in certain implementations, the bubble-detection system 106 utilizes the image-machine-learning model 608 to identify or generate a training data set 620 for a bubble-detection-machine-learning model. For instance, in some cases, the bubble-detection system 106 identifies, as part of the training data set 620, nucleobase calls from the nucleobase calls 602a-602n for which the image-machine-learning model 608 correctly detects the presence (or the absence) of bubbles within sections (e.g., tiles or sub-tiles) of a nucleotide-sample slide depicted by the corresponding spatial images. Having identified such nucleobase calls from BCL or BAM files for the training data set 620, the bubble-detection system 106 likewise identifies, for the training data set 620, corresponding ground-truth classification labels from the ground-truth classification labels 604a-604n that correctly indicate the presence (or the absence) of bubbles. In some instances, the ground-truth classification labels are modified to correctly indicate the presence (or the absence) of bubbles within sections of a nucleotide-sample slide—for the corresponding nucleobase calls selected for inclusion within the training data set 620. As shown in FIG. 6A, the bubble-detection system 106 selects, to include within the training data set 620, combinations of (i) nucleobase calls, (ii) corresponding quality metrics, and (iii) corresponding ground-truth-classification labels for spatial images that produced a correctly detected presence or absence of a bubble from the image-machine-learning model 608.

In the alternative to using the image-machine-learning model 608 to identify the training data set 620, in some embodiments, the bubble-detection system 106 identifies, as part of the training data set 620, nucleobase calls from the nucleobase calls 602a-602n for which researchers correctly detect the presence (or the absence) of bubbles within sections (e.g., tiles or sub-tiles) of a nucleotide-sample slide depicted by the corresponding spatial images. In other words, in some embodiments, the bubble-detection system 106 uses the spatial images 606a-606n identified by humans with technical expertise (rather than the image-machine-learning model 608) to select nucleobase calls from the nucleobase calls 602a-602n for inclusion within the training data set 620. In some such cases, the bubble-detection system 106 uses the nucleobase calls from BCL or BAM files corresponding to such spatial images with sections containing bubbles (or no bubbles) identified by humans. As shown in FIG. 6A, the bubble-detection system 106 alternatively selects, to include within the training data set 620, combinations of (i) nucleobase calls, (ii) corresponding quality metrics, and (iii) corresponding ground-truth-classification labels for spatial images that technicians or researchers correctly detected the presence or absence of a bubble.

Regardless of how the training data set 620 is selected, as further shown in FIG. 6A, the bubble-detection system 106 utilizes the training data set 620 to train a bubble-detection-machine-learning model 622 (e.g., the bubble-detection-neural network 500 illustrated in FIG. 5). As indicated above, in some cases, the bubble-detection system 106 utilizes training input matrices from the training data set 620 comprising a first subset of nucleobase calls corresponding to at least one nucleobase and a second subset of nucleobase calls satisfying a threshold quality metric. More specifically, the bubble-detection system 106 generates training input matrices comprising a subset (e.g., percentage) of adenine calls, a subset of guanine calls, and a subset of nucleobase calls satisfying a threshold quality metric (e.g., Q30) from the training data set 620. In such embodiments, the bubble-detection-machine-learning model 622 is trained to generate error classifications (e.g., air bubble, oil bubble, etc.).

In the alternative to inputting such subsets of nucleobase calls from the training data set 620, in some embodiments, the bubble-detection system 106 inputs, into the bubble-detection-machine-learning model 622, nucleobase calls arranged according to clusters within sections of a nucleotide-sample slide and corresponding quality metrics. By using nucleobase calls arranged according to cluster as inputs for the bubble-detection-machine-learning model 622, the bubble-detection system 106 can identify patterns of nucleobase calls indicating a presence or absence of a bubble. For instance, such nucleobase calls may reflect a pattern of nucleobase calls (e.g., a circular pattern of A calls or circular pattern of G calls) indicating a presence of a bubble within a section (e.g., tile or sub-tile) of a nucleotide-sample slide.

Regardless of the form of the training data set 620, as indicated by FIG. 6A, the bubble-detection system 106 uses the training data set 620 to train the bubble-detection-machine-learning model 622. In an initial training iteration, for example, the bubble-detection system 106 inputs an input matrix comprising a first subset of nucleobase calls corresponding to at least one nucleobase and a second subset of nucleobase calls satisfying a threshold quality metric from the training data set 620. Alternatively, the bubble-detection system 106 inputs nucleobase calls arranged according to clusters within sections of a nucleotide-sample slide and corresponding quality metrics from the training data set 620.

Based on the input data, the bubble-detection-machine-learning model 622 determines predicted classification labels 624 indicating a presence or absence of a bubble. In some cases, the predicted classification labels 624 indicate a presence or absence of a particulate type of bubble (e.g., air bubble, oil bubble) and a particular section of a nucleotide-sample slide. For instance, the predicted classification labels 624 may indicate the presence or absence of a bubble within a tile or sub-tile of a flow cell. As indicated above, in one or more embodiments, the bubble-detection system 106 determines confidence scores corresponding to individual classifications from the predicted classification labels 624. Accordingly, the bubble-detection system 106 can determine the predicted classification labels 624 based on the generated confidence scores.

As further illustrated in FIG. 6A, the bubble-detection system 106 uses a loss function 626 to compare the predicted classification labels 624 with the corresponding ground-truth classification labels from the training data set 620. In some implementations, the ground-truth classification labels from the training data set 620 comprise ground-truth bubble classifications and bubble locations corresponding to the input nucleobase-call data and quality metrics. Similar to the training process described above, for instance, the ground-truth classification labels can indicate (i) a particular section or sub-section of a nucleotide-sample slide that contains a bubble or a portion of a bubble and (ii) a particular section or sub-section of a nucleotide-sample slide that contains no bubble or no portion of a bubble.

Depending on the form of the bubble-detection-machine-learning model 622, the bubble-detection system 106 can use a variety of loss functions for the loss function 626. In certain embodiments, the bubble-detection system 106 uses a cross-entropy-loss function (e.g., for a CNN). But any suitable loss function may be used as the loss function 626. Regardless of the form of the loss function 626, the bubble-detection system 106 determines a loss 628a from the loss function 626 based on a comparison of the predicted classification labels 624 with the corresponding ground-truth classification labels from the training data set 620. Indeed, in certain implementations, the loss 628a may include separate losses for a particular section of a nucleotide-sample slide (e.g., tile or sub-tile).

Based on the determined loss 628a from the loss function 626, the bubble-detection system 106 subsequently adjusts parameters of the bubble-detection-machine-learning model 622. By adjusting the parameters, the bubble-detection system 106 increases the accuracy with which the bubble-detection-machine-learning model 622 determines the presence and location of bubbles over multiple training iterations. Indeed, as further shown in FIG. 6A, the bubble-detection system 106 performs subsequent training iterations. As suggested by FIG. 6A, in some embodiments, the bubble-detection system 106 iteratively inputs data derived from nucleobase calls and quality metrics from the training data set 620 into the bubble-detection-machine-learning model 622 to generate predicted classification labels, iteratively compares the predicted classification labels to corresponding ground-truth classification labels from the training data set 620 to determine losses 628a-628n, and iteratively adjusts the parameters of the bubble-detection-machine-learning model 622. In some cases, the bubble-detection system 106 performs training iterations until the parameters (e.g., value or weights) of the bubble-detection-machine-learning model 622 do not change significantly across training iterations or otherwise satisfy a convergence criteria.

In addition to generating predicted classification labels, in some implementations, the bubble-detection system 106 trains the bubble-detection-machine-learning model 622 to infer the size of a bubble. In particular, the bubble-detection-machine-learning model 622 can extract features from nucleobase calls of the training data set 620 to predict the size of an identified bubble. To illustrate, the bubble-detection system 106 can train the bubble-detection-machine-learning model 622 to determine the diameter of predicted bubbles based on the spatial data derived from the nucleobase calls and the quality metrics. Alternatively, the bubble-detection system 106 trains the bubble-detection-machine-learning model 622 to determine the size of a bubble based on the intensity of a spike or dip in the percent of nucleobase calls or percent Q30. Thus, the bubble-detection system 106 can train the bubble-detection-machine-learning model 622 to generate a predicted bubble size based on an analysis of input data.

As mentioned previously, in some embodiments, the bubble-detection system 106 reduces the quality metric (e.g., the Q score) for a given read, cycle, section, or sub-section of a nucleotide-sample slide based on determining the presence of a bubble. In some embodiments, the bubble-detection system 106 reduces the quality metric based on a size or diameter of a detected bubble. For example, the bubble-detection system 106 generates a predicted diameter of a detected bubble using the bubble-detection-machine-learning model 622 and associates greater diameter sizes with greater reductions in quality metrics. Furthermore, in some embodiments, the bubble-detection system 106 determines a threshold bubble diameter value below which the bubble-detection system 106 does not alter the quality metric. In particular, the bubble-detection system 106 may determine that smaller bubbles have a negligible impact on read quality.

As described previously, the bubble-detection system 106 can identify or generate a spatial image comprising spatial patterns corresponding to nucleobase calls. FIG. 6B illustrates an example spatial image in accordance with one or more embodiments. In particular, FIG. 6B illustrates a spatial image 636 comprising a tile 640 having a spatial pattern 638. As illustrated, the bubble-detection system 106 constructs the spatial image 636 using nucleobase calls 642. Alternatively, the bubble-detection system 106 receives the spatial image 636 as one for which a technician or researcher identifies a bubble within the tile 640.

As mentioned previously, in some embodiments, the bubble-detection system 106 can analyze shapes of spatial patterns identified within the spatial image 636 to determine the presence or absence of bubbles or other artifacts. As indicated by FIG. 6B, for instance, the bubble-detection-machine-learning model 622 may detect a circular pattern of G calls as representing a bubble. Indeed, in certain implementations, the bubble-detection system 106 associates circular spatial patterns of particular nucleobase calls (e.g., A calls or G calls) with bubbles and non-circular or alternative spatial patterns with other types of artifacts. As for the latter artifacts, for example, the bubble-detection system 106 may associate alternative spatial patterns with artifacts, such as a low occupancy region or an amplicon region.

To help visualize a real-life example of a bubble within a nucleotide-sample slide, the disclosure includes FIG. 6C. In particular, FIG. 6C illustrates a sequencing run image 650 depicting a portion of a flow cell 658 comprising tiles, including tiles 656a-656c. As illustrated in FIG. 6C, the sequencing run image 650 depicts dark circular regions corresponding to bubbles 654a-654c that go across or are present within various tiles. For example, FIG. 6C illustrates the bubble 654b spanning the tile 656a and the tile 656b while the bubble 654c is contained within the tile 656c.

FIG. 6C illustrates an example sequencing run image that demonstrates the appearance of bubbles on a flow cell. As described previously, accessing, storing, and processing image data is computationally expensive and often impractical. Thus, in some implementations, the bubble-detection system 106 does not access the sequencing run image 650 and instead accesses and processes nucleobase-call data and quality metrics (from various file types) to confirm the presence or absence of bubbles, as described above.

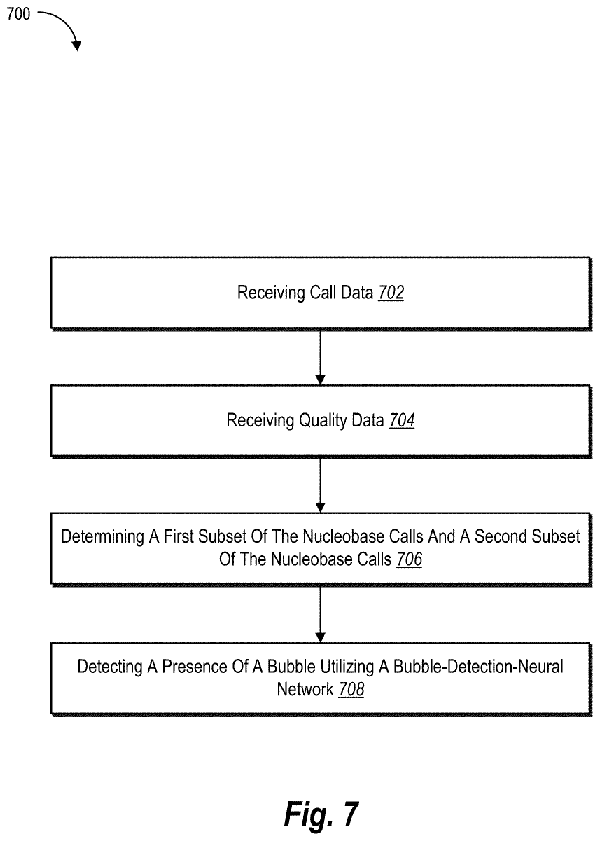

FIGS. 1-6B, the corresponding text, and the examples provide a number of different methods, systems, devices, and non-transitory computer-readable media of the bubble-detection system 106. In addition to the foregoing, one or more embodiments can also be described in terms of flowcharts comprising acts for accomplishing a particular result, such as the flowchart of acts shown in FIG. 7. Additionally, the acts described herein may be repeated or performed in parallel with one another or in parallel with different instances of the same or similar acts.

FIG. 7 illustrates a flowchart of a series of acts 700 for detecting a presence of a bubble within a nucleotide-sample slide. While FIG. 7 illustrates acts according to one embodiment, alternative embodiments may omit, add to, reorder and/or modify any of the acts shown in FIG. 7. The acts of FIG. 7 can be performed as part of a method. Alternatively, a non-transitory computer-readable medium can comprise instructions that, when executed by one or more processors, cause a computing device to perform the acts of FIG. 7. In some embodiments, a system can perform the acts of FIG. 7.

In one or more embodiments, the series of acts 700 is implemented on one or more computing devices, such as the computing device illustrated in FIG. 8. In addition, in some embodiments, the series of acts 700 is implemented in a digital environment for sequencing nucleic-acid polymers. For example, the series of acts 700 is implemented on a computing device having a memory that includes a bubble-detection-machine-learning model. In some embodiments, the memory also stores training data including ground truth classifications and training input matrices.

As illustrated in FIG. 7, the series of acts 700 includes an act 702 of receiving call data. In particular, the act 702 comprises receiving, for a nucleotide-sample slide, call data comprising nucleobase calls for cycles of sequencing a nucleic-acid polymer. In some embodiments, the act 702 further comprises receive the call data comprising the nucleobase calls based on: one-channel-intensity data comprising a single image for each section of the nucleotide-sample slide for a given cycle of sequencing the nucleic-acid polymer; two-channel data comprising two images for each section of the nucleotide-sample slide for the given cycle of sequencing the nucleic-acid polymer; or four-channel data comprising four images for each section of the nucleotide-sample slide for the given cycle of sequencing the nucleic-acid polymer.

The series of acts 700 illustrated in FIG. 7 includes an act 704 of receiving quality data. In particular, the act 704 comprises receiving, for the nucleotide-sample slide, quality data comprising quality metrics that estimate errors in the nucleobase calls for the cycles.

The series of acts 700 includes an act 706 of determining a first subset of the nucleobase calls and a second subset of the nucleobase calls. In particular, the act 706 comprises determining, from the nucleobase calls for the cycles, a first subset of the nucleobase calls corresponding to at least one nucleobase and a second subset of the nucleobase calls satisfying a threshold quality metric for the quality metrics. In some embodiments, the act 706 further comprises determining the first subset of the nucleobase calls corresponding to the at least one nucleobase by determining at least one of a subset of adenine calls, a subset of thymine calls, a subset of cytosine calls, or a subset of guanine calls for the cycles of sequencing the nucleic-acid polymer.

As further illustrated in FIG. 7, the series of acts 700 includes an act 708 of detecting a presence of a bubble utilizing a bubble-detection-neural network. In particular, the act 708 comprises detecting a presence of a bubble within the nucleotide-sample slide utilizing a bubble-detection-machine-learning model based on the first subset of the nucleobase calls and the second subset of the nucleobase calls. Additionally, in one or more embodiments, the bubble-detection-neural network comprises at least one of a Support Vector Machine or an Adaptive Boosting machine learning model.

In some implementations, the act 708 further comprises detecting the presence of the bubble utilizing the bubble-detection-machine-learning model by extracting, utilizing layers of the bubble-detection-machine-learning model, features from an input matrix comprising the subset of adenine calls, the subset of guanine calls, and the second subset of the nucleobase calls satisfying the threshold quality metric for the cycles of sequencing the nucleic-acid polymer. Furthermore, in one or more embodiments, the act 708 comprises detecting the presence of the bubble by detecting at least one of an air bubble, an oil bubble, or a ghost bubble within the nucleotide-sample slide. Additionally, in some embodiments, the bubble-detection-machine-learning model comprises a convolutional neural network comprising feature extraction layers, classification layers, and an adaptive max pooling layer between the feature extraction layers and the classification layers.

In one or more embodiments, the act 708 further includes the additional act of detecting the presence of the bubble by: generating, utilizing the bubble-detection-machine-learning model, a probability that a section of the nucleotide-sample slide contains the bubble; and determining that the probability satisfies a threshold value indicating the presence of the bubble.

In some embodiments, the series of acts 700 includes the additional acts of receiving the call data and the quality data for a section of the nucleotide-sample slide and detecting the presence of the bubble within the section of the nucleotide-sample slide. More specifically, in some embodiments, the additional acts further comprises detecting the presence of the bubble within the section of the nucleotide-sample slide by detecting the bubble within a tile of a flow cell.

Additionally, in some implementations, the series of acts 700 further include the additional act of determining the presence of the bubble during one or more cycles of the cycles of sequencing the nucleic-acid polymer.

Furthermore, in one or more embodiments, the series of acts 700 further comprise the act of providing, for display on the computing device, an alert indicating the presence of the bubble within the nucleotide-sample slide.

Additionally, in some implementations, the series of acts 700 include an additional act of determining the presence of the bubble during a cycle of the cycles of sequencing the nucleic-acid polymer.

The methods described herein can be used in conjunction with a variety of nucleic acid sequencing techniques. Particularly applicable techniques are those wherein nucleic acids are attached at fixed locations in an array such that their relative positions do not change and wherein the array is repeatedly imaged. Embodiments in which images are obtained in different color channels, for example, coinciding with different labels used to distinguish one nucleotide base type from another are particularly applicable. In some embodiments, the process to determine the nucleotide sequence of a target nucleic acid (i.e., a nucleic-acid polymer) can be an automated process. Preferred embodiments include sequencing-by-synthesis (SBS) techniques.

SBS techniques generally involve the enzymatic extension of a nascent nucleic acid strand through the iterative addition of nucleotides against a template strand. In traditional methods of SBS, a single nucleotide monomer may be provided to a target nucleotide in the presence of a polymerase in each delivery. However, in the methods described herein, more than one type of nucleotide monomer can be provided to a target nucleic acid in the presence of a polymerase in a delivery.

The SBS techniques described below can utilize single-read sequencing or paired-end sequencing. In single-rea sequencing, the sequencing device reads a fragment from one end to another to generate the sequence of base pairs. In contrast, during paired-end sequencing, the sequencing device begins at one read, finishes reading a specified read length in the same direction, and begins another read from the opposite end of the fragment.

SBS can utilize nucleotide monomers that have a terminator moiety or those that lack any terminator moieties. Methods utilizing nucleotide monomers lacking terminators include, for example, pyrosequencing and sequencing using γ-phosphate-labeled nucleotides, as set forth in further detail below. In methods using nucleotide monomers lacking terminators, the number of nucleotides added in each cycle is generally variable and dependent upon the template sequence and the mode of nucleotide delivery. For SBS techniques that utilize nucleotide monomers having a terminator moiety, the terminator can be effectively irreversible under the sequencing conditions used as is the case for traditional Sanger sequencing which utilizes dideoxynucleotides, or the terminator can be reversible as is the case for sequencing methods developed by Solexa (now Illumina, Inc.).

SBS techniques can utilize nucleotide monomers that have a label moiety or those that lack a label moiety. Accordingly, incorporation events can be detected based on a characteristic of the label, such as fluorescence of the label; a characteristic of the nucleotide monomer such as molecular weight or charge; a byproduct of incorporation of the nucleotide, such as release of pyrophosphate; or the like. In embodiments, where two or more different nucleotides are present in a sequencing reagent, the different nucleotides can be distinguishable from each other, or alternatively, the two or more different labels can be the indistinguishable under the detection techniques being used. For example, the different nucleotides present in a sequencing reagent can have different labels and they can be distinguished using appropriate optics as exemplified by the sequencing methods developed by Solexa (now Illumina, Inc.).

Preferred embodiments include pyrosequencing techniques. Pyrosequencing detects the release of inorganic pyrophosphate (PPi) as particular nucleotides are incorporated into the nascent strand (Ronaghi, M., Karamohamed, S., Pettersson, B., Uhlen, M. and Nyren, P. (1996) “Real-time DNA sequencing using detection of pyrophosphate release.” Analytical Biochemistry 242(1), 84-9; Ronaghi, M. (2001) “Pyrosequencing sheds light on DNA sequencing.” Genome Res. 11(1), 3-11; Ronaghi, M., Uhlen, M. and Nyren, P. (1998) “A sequencing method based on real-time pyrophosphate.” Science 281(5375), 363; U.S. Pat. Nos. 6,210,891; 6,258,568 and 6,274,320, the disclosures of which are incorporated herein by reference in their entireties). In pyrosequencing, released PPi can be detected by being immediately converted to adenosine triphosphate (ATP) by ATP sulfurylase, and the level of ATP generated is detected via luciferase-produced photons. The nucleic acids to be sequenced can be attached to features in an array and the array can be imaged to capture the chemiluminescent signals that are produced due to incorporation of a nucleotides at the features of the array. An image can be obtained after the array is treated with a particular nucleotide type (e.g. A, T, C or G). Images obtained after addition of each nucleotide type will differ with regard to which features in the array are detected. These differences in the image reflect the different sequence content of the features on the array. However, the relative locations of each feature will remain unchanged in the images. The images can be stored, processed and analyzed using the methods set forth herein. For example, images obtained after treatment of the array with each different nucleotide type can be handled in the same way as exemplified herein for images obtained from different detection channels for reversible terminator-based sequencing methods.

In another exemplary type of SBS, cycle sequencing is accomplished by stepwise addition of reversible terminator nucleotides containing, for example, a cleavable or photobleachable dye label as described, for example, in WO 04/018497 and U.S. Pat. No. 7,057,026, the disclosures of which are incorporated herein by reference. This approach is being commercialized by Solexa (now Illumina Inc.), and is also described in WO 91/06678 and WO 07/123,744, each of which is incorporated herein by reference. The availability of fluorescently-labeled terminators in which both the termination can be reversed and the fluorescent label cleaved facilitates efficient cyclic reversible termination (CRT) sequencing. Polymerases can also be co-engineered to efficiently incorporate and extend from these modified nucleotides.

Preferably in reversible terminator-based sequencing embodiments, the labels do not substantially inhibit extension under SBS reaction conditions. However, the detection labels can be removable, for example, by cleavage or degradation. Images can be captured following incorporation of labels into arrayed nucleic acid features. In particular embodiments, each cycle involves simultaneous delivery of four different nucleotide types to the array and each nucleotide type has a spectrally distinct label. Four images can then be obtained, each using a detection channel that is selective for one of the four different labels. Alternatively, different nucleotide types can be added sequentially and an image of the array can be obtained between each addition step. In such embodiments, each image will show nucleic acid features that have incorporated nucleotides of a particular type. Different features will be present or absent in the different images due the different sequence content of each feature. However, the relative position of the features will remain unchanged in the images. Images obtained from such reversible terminator-SBS methods can be stored, processed and analyzed as set forth herein. Following the image capture step, labels can be removed and reversible terminator moieties can be removed for subsequent cycles of nucleotide addition and detection. Removal of the labels after they have been detected in a particular cycle and prior to a subsequent cycle can provide the advantage of reducing background signal and crosstalk between cycles. Examples of useful labels and removal methods are set forth below.

In particular embodiments some or all of the nucleotide monomers can include reversible terminators. In such embodiments, reversible terminators/cleavable fluors can include fluor linked to the ribose moiety via a 3′ ester linkage (Metzker, Genome Res. 15:1767-1776 (2005), which is incorporated herein by reference). Other approaches have separated the terminator chemistry from the cleavage of the fluorescence label (Ruparel et al., Proc Natl Acad Sci USA 102: 5932-7 (2005), which is incorporated herein by reference in its entirety). Ruparel et al described the development of reversible terminators that used a small 3′ allyl group to block extension, but could easily be deblocked by a short treatment with a palladium catalyst. The fluorophore was attached to the base via a photocleavable linker that could easily be cleaved by a 30 second exposure to long wavelength UV light. Thus, either disulfide reduction or photocleavage can be used as a cleavable linker. Another approach to reversible termination is the use of natural termination that ensues after placement of a bulky dye on a dNTP. The presence of a charged bulky dye on the dNTP can act as an effective terminator through steric and/or electrostatic hindrance. The presence of one incorporation event prevents further incorporations unless the dye is removed. Cleavage of the dye removes the fluor and effectively reverses the termination. Examples of modified nucleotides are also described in U.S. Pat. Nos. 7,427,673, and 7,057,026, the disclosures of which are incorporated herein by reference in their entireties.

Additional exemplary SBS systems and methods which can be utilized with the methods and systems described herein are described in U.S. Patent Application Publication No. 2007/0166705, U.S. Patent Application Publication No. 2006/0188901, U.S. Pat. No. 7,057,026, U.S. Patent Application Publication No. 2006/0240439, U.S. Patent Application Publication No. 2006/0281109, PCT Publication No. WO 05/065814, U.S. Patent Application Publication No. 2005/0100900, PCT Publication No. WO 06/064199, PCT Publication No. WO 07/010,251, U.S. Patent Application Publication No. 2012/0270305 and U.S. Patent Application Publication No. 2013/0260372, the disclosures of which are incorporated herein by reference in their entireties.

Some embodiments can utilize detection of four different nucleotides using fewer than four different labels. For example, SBS can be performed utilizing methods and systems described in the incorporated materials of U.S. Patent Application Publication No. 2013/0079232. As a first example, a pair of nucleotide types can be detected at the same wavelength, but distinguished based on a difference in intensity for one member of the pair compared to the other, or based on a change to one member of the pair (e.g. via chemical modification, photochemical modification or physical modification) that causes apparent signal to appear or disappear compared to the signal detected for the other member of the pair. As a second example, three of four different nucleotide types can be detected under particular conditions while a fourth nucleotide type lacks a label that is detectable under those conditions, or is minimally detected under those conditions (e.g., minimal detection due to background fluorescence, etc.). Incorporation of the first three nucleotide types into a nucleic acid can be determined based on presence of their respective signals and incorporation of the fourth nucleotide type into the nucleic acid can be determined based on absence or minimal detection of any signal. As a third example, one nucleotide type can include label(s) that are detected in two different channels, whereas other nucleotide types are detected in no more than one of the channels. The aforementioned three exemplary configurations are not considered mutually exclusive and can be used in various combinations. An exemplary embodiment that combines all three examples, is a fluorescent-based SBS method that uses a first nucleotide type that is detected in a first channel (e.g. dATP having a label that is detected in the first channel when excited by a first excitation wavelength), a second nucleotide type that is detected in a second channel (e.g. dCTP having a label that is detected in the second channel when excited by a second excitation wavelength), a third nucleotide type that is detected in both the first and the second channel (e.g. dTTP having at least one label that is detected in both channels when excited by the first and/or second excitation wavelength) and a fourth nucleotide type that lacks a label that is not, or minimally, detected in either channel (e.g. dGTP having no label).

Further, as described in the incorporated materials of U.S. Patent Application Publication No. 2013/0079232, sequencing data can be obtained using a single channel. In such so-called one-dye sequencing approaches, the first nucleotide type is labeled but the label is removed after the first image is generated, and the second nucleotide type is labeled only after a first image is generated. The third nucleotide type retains its label in both the first and second images, and the fourth nucleotide type remains unlabeled in both images.

Some embodiments can utilize sequencing by ligation techniques. Such techniques utilize DNA ligase to incorporate oligonucleotides and identify the incorporation of such oligonucleotides. The oligonucleotides typically have different labels that are correlated with the identity of a particular nucleotide in a sequence to which the oligonucleotides hybridize. As with other SBS methods, images can be obtained following treatment of an array of nucleic acid features with the labeled sequencing reagents. Each image will show nucleic acid features that have incorporated labels of a particular type. Different features will be present or absent in the different images due the different sequence content of each feature, but the relative position of the features will remain unchanged in the images. Images obtained from ligation-based sequencing methods can be stored, processed and analyzed as set forth herein. Exemplary SBS systems and methods which can be utilized with the methods and systems described herein are described in U.S. Pat. Nos. 6,969,488, 6,172,218, and 6,306,597, the disclosures of which are incorporated herein by reference in their entireties.

Some embodiments can utilize nanopore sequencing (Deamer, D. W. & Akeson, M. “Nanopores and nucleic acids: prospects for ultrarapid sequencing.” Trends Biotechnol. 18, 147-151 (2000); Deamer, D. and D. Branton, “Characterization of nucleic acids by nanopore analysis”. Acc. Chem. Res. 35:817-825 (2002); Li, J., M. Gershow, D. Stein, E. Brandin, and J. A. Golovchenko, “DNA molecules and configurations in a solid-state nanopore microscope” Nat. Mater. 2:611-615 (2003), the disclosures of which are incorporated herein by reference in their entireties). In such embodiments, the target nucleic acid passes through a nanopore. The nanopore can be a synthetic pore or biological membrane protein, such as α-hemolysin. As the target nucleic acid passes through the nanopore, each base-pair can be identified by measuring fluctuations in the electrical conductance of the pore. (U.S. Pat. No. 7,001,792; Soni, G. V. & Meller, “A. Progress toward ultrafast DNA sequencing using solid-state nanopores.” Clin. Chem. 53, 1996-2001 (2007); Healy, K. “Nanopore-based single-molecule DNA analysis.” Nanomed. 2, 459-481 (2007); Cockroft, S. L., Chu, J., Amorin, M. & Ghadiri, M. R. “A single-molecule nanopore device detects DNA polymerase activity with single-nucleotide resolution.” J. Am. Chem. Soc. 130, 818-820 (2008), the disclosures of which are incorporated herein by reference in their entireties). Data obtained from nanopore sequencing can be stored, processed and analyzed as set forth herein. In particular, the data can be treated as an image in accordance with the exemplary treatment of optical images and other images that is set forth herein.

Some embodiments can utilize methods involving the real-time monitoring of DNA polymerase activity. Nucleotide incorporations can be detected through fluorescence resonance energy transfer (FRET) interactions between a fluorophore-bearing polymerase and γ-phosphate-labeled nucleotides as described, for example, in U.S. Pat. Nos. 7,329,492 and 7,211,414 (each of which is incorporated herein by reference) or nucleotide incorporations can be detected with zero-mode waveguides as described, for example, in U.S. Pat. No. 7,315,019 (which is incorporated herein by reference) and using fluorescent nucleotide analogs and engineered polymerases as described, for example, in U.S. Pat. No. 7,405,281 and U.S. Patent Application Publication No. 2008/0108082 (each of which is incorporated herein by reference). The illumination can be restricted to a zeptoliter-scale volume around a surface-tethered polymerase such that incorporation of fluorescently labeled nucleotides can be observed with low background (Levene, M. J. et al. “Zero-mode waveguides for single-molecule analysis at high concentrations.” Science 299, 682-686 (2003); Lundquist, P. M. et al. “Parallel confocal detection of single molecules in real time.” Opt. Lett. 33, 1026-1028 (2008); Korlach, J. et al. “Selective aluminum passivation for targeted immobilization of single DNA polymerase molecules in zero-mode waveguide nano structures.” Proc. Natl. Acad. Sci. USA 105, 1176-1181 (2008), the disclosures of which are incorporated herein by reference in their entireties). Images obtained from such methods can be stored, processed and analyzed as set forth herein.

Some SBS embodiments include detection of a proton released upon incorporation of a nucleotide into an extension product. For example, sequencing based on detection of released protons can use an electrical detector and associated techniques that are commercially available from Ion Torrent (Guilford, CT, a Life Technologies subsidiary) or sequencing methods and systems described in US 2009/0026082 A1; US 2009/0127589 A1; US 2010/0137143 A1; or US 2010/0282617 A1, each of which is incorporated herein by reference. Methods set forth herein for amplifying target nucleic acids using kinetic exclusion can be readily applied to substrates used for detecting protons. More specifically, methods set forth herein can be used to produce clonal populations of amplicons that are used to detect protons.

The above SBS methods can be advantageously carried out in multiplex formats such that multiple different target nucleic acids are manipulated simultaneously. In particular embodiments, different target nucleic acids can be treated in a common reaction vessel or on a surface of a particular substrate. This allows convenient delivery of sequencing reagents, removal of unreacted reagents and detection of incorporation events in a multiplex manner. In embodiments using surface-bound target nucleic acids, the target nucleic acids can be in an array format. In an array format, the target nucleic acids can be typically bound to a surface in a spatially distinguishable manner. The target nucleic acids can be bound by direct covalent attachment, attachment to a bead or other particle or binding to a polymerase or other molecule that is attached to the surface. The array can include a single copy of a target nucleic acid at each site (also referred to as a feature) or multiple copies having the same sequence can be present at each site or feature. Multiple copies can be produced by amplification methods such as, bridge amplification or emulsion PCR as described in further detail below.

The methods set forth herein can use arrays having features at any of a variety of densities including, for example, at least about 10 features/cm², 100 features/cm², 500 features/cm², 1,000 features/cm², 5,000 features/cm², 10,000 features/cm², 50,000 features/cm², 100,000 features/cm², 1,000,000 features/cm², 5,000,000 features/cm², or higher.

An advantage of the methods set forth herein is that they provide for rapid and efficient detection of a plurality of target nucleic acid in parallel. Accordingly the present disclosure provides integrated systems capable of preparing and detecting nucleic acids using techniques known in the art such as those exemplified above. Thus, an integrated system of the present disclosure can include fluidic components capable of delivering amplification reagents and/or sequencing reagents to one or more immobilized DNA fragments, the system comprising components such as pumps, valves, reservoirs, fluidic lines and the like. A flow cell can be configured and/or used in an integrated system for detection of target nucleic acids. Exemplary flow cells are described, for example, in US 2010/0111768 A1 and U.S. Ser. No. 13/273,666, each of which is incorporated herein by reference. As exemplified for flow cells, one or more of the fluidic components of an integrated system can be used for an amplification method and for a detection method. Taking a nucleic acid sequencing embodiment as an example, one or more of the fluidic components of an integrated system can be used for an amplification method set forth herein and for the delivery of sequencing reagents in a sequencing method such as those exemplified above. Alternatively, an integrated system can include separate fluidic systems to carry out amplification methods and to carry out detection methods. Examples of integrated sequencing systems that are capable of creating amplified nucleic acids and also determining the sequence of the nucleic acids include, without limitation, the MiSeg™ platform (Illumina, Inc., San Diego, Calif.) and devices described in U.S. Ser. No. 13/273,666, which is incorporated herein by reference.

The sequencing system described above sequences nucleic-acid polymers present in samples received by a sequencing device. As defined herein, “sample” and its derivatives, is used in its broadest sense and includes any specimen, culture and the like that is suspected of including a target. In some embodiments, the sample comprises DNA, RNA, PNA, LNA, chimeric or hybrid forms of nucleic acids. The sample can include any biological, clinical, surgical, agricultural, atmospheric or aquatic-based specimen containing one or more nucleic acids. The term also includes any isolated nucleic acid sample such a genomic DNA, fresh-frozen or formalin-fixed paraffin-embedded nucleic acid specimen. It is also envisioned that the sample can be from a single individual, a collection of nucleic acid samples from genetically related members, nucleic acid samples from genetically unrelated members, nucleic acid samples (matched) from a single individual such as a tumor sample and normal tissue sample, or sample from a single source that contains two distinct forms of genetic material such as maternal and fetal DNA obtained from a maternal subject, or the presence of contaminating bacterial DNA in a sample that contains plant or animal DNA. In some embodiments, the source of nucleic acid material can include nucleic acids obtained from a newborn, for example as typically used for newborn screening.

The nucleic acid sample can include high molecular weight material such as genomic DNA (gDNA). The sample can include low molecular weight material such as nucleic acid molecules obtained from FFPE or archived DNA samples. In another embodiment, low molecular weight material includes enzymatically or mechanically fragmented DNA. The sample can include cell-free circulating DNA. In some embodiments, the sample can include nucleic acid molecules obtained from biopsies, tumors, scrapings, swabs, blood, mucus, urine, plasma, semen, hair, laser capture micro-dissections, surgical resections, and other clinical or laboratory obtained samples. In some embodiments, the sample can be an epidemiological, agricultural, forensic or pathogenic sample. In some embodiments, the sample can include nucleic acid molecules obtained from an animal such as a human or mammalian source. In another embodiment, the sample can include nucleic acid molecules obtained from a non-mammalian source such as a plant, bacteria, virus or fungus. In some embodiments, the source of the nucleic acid molecules may be an archived or extinct sample or species.

Further, the methods and compositions disclosed herein may be useful to amplify a nucleic acid sample having low-quality nucleic acid molecules, such as degraded and/or fragmented genomic DNA from a forensic sample. In one embodiment, forensic samples can include nucleic acids obtained from a crime scene, nucleic acids obtained from a missing persons DNA database, nucleic acids obtained from a laboratory associated with a forensic investigation or include forensic samples obtained by law enforcement agencies, one or more military services or any such personnel. The nucleic acid sample may be a purified sample or a crude DNA containing lysate, for example derived from a buccal swab, paper, fabric or other substrate that may be impregnated with saliva, blood, or other bodily fluids. As such, in some embodiments, the nucleic acid sample may comprise low amounts of, or fragmented portions of DNA, such as genomic DNA. In some embodiments, target sequences can be present in one or more bodily fluids including but not limited to, blood, sputum, plasma, semen, urine and serum. In some embodiments, target sequences can be obtained from hair, skin, tissue samples, autopsy or remains of a victim. In some embodiments, nucleic acids including one or more target sequences can be obtained from a deceased animal or human. In some embodiments, target sequences can include nucleic acids obtained from non-human DNA such a microbial, plant or entomological DNA. In some embodiments, target sequences or amplified target sequences are directed to purposes of human identification. In some embodiments, the disclosure relates generally to methods for identifying characteristics of a forensic sample. In some embodiments, the disclosure relates generally to human identification methods using one or more target specific primers disclosed herein or one or more target specific primers designed using the primer design criteria outlined herein. In one embodiment, a forensic or human identification sample containing at least one target sequence can be amplified using any one or more of the target-specific primers disclosed herein or using the primer criteria outlined herein.

The components of the bubble-detection system 106 can include software, hardware, or both. For example, the components of the bubble-detection system 106 can include one or more instructions stored on a computer-readable storage medium and executable by processors of one or more computing devices (e.g., the user client device 108). When executed by the one or more processors, the computer-executable instructions of the bubble-detection system 106 can cause the computing devices to perform the bubble detection methods described herein. Alternatively, the components of the bubble-detection system 106 can comprise hardware, such as special purpose processing devices to perform a certain function or group of functions. Additionally, or alternatively, the components of the bubble-detection system 106 can include a combination of computer-executable instructions and hardware.

Furthermore, the components of the bubble-detection system 106 performing the functions described herein with respect to the bubble-detection system 106 may, for example, be implemented as part of a stand-alone application, as a module of an application, as a plug-in for applications, as a library function or functions that may be called by other applications, and/or as a cloud-computing model. Thus, components of the bubble-detection system 106 may be implemented as part of a stand-alone application on a personal computing device or a mobile device. Additionally, or alternatively, the components of the bubble-detection system 106 may be implemented in any application that provides sequencing services including, but not limited to Illumina BaseSpace, Illumina DRAGEN, or Illumina TruSight software. “Illumina,” “BaseSpace,” “DRAGEN,” and “TruSight,” are either registered trademarks or trademarks of Illumina, Inc. in the United States and/or other countries.

Embodiments of the present disclosure may comprise or utilize a special purpose or general-purpose computer including computer hardware, such as, for example, one or more processors and system memory, as discussed in greater detail below. Embodiments within the scope of the present disclosure also include physical and other computer-readable media for carrying or storing computer-executable instructions and/or data structures. In particular, one or more of the processes described herein may be implemented at least in part as instructions embodied in a non-transitory computer-readable medium and executable by one or more computing devices (e.g., any of the media content access devices described herein). In general, a processor (e.g., a microprocessor) receives instructions, from a non-transitory computer-readable medium, (e.g., a memory, etc.), and executes those instructions, thereby performing one or more processes, including one or more of the processes described herein.

Computer-readable media can be any available media that can be accessed by a general purpose or special purpose computer system. Computer-readable media that store computer-executable instructions are non-transitory computer-readable storage media (devices). Computer-readable media that carry computer-executable instructions are transmission media. Thus, by way of example, and not limitation, embodiments of the disclosure can comprise at least two distinctly different kinds of computer-readable media: non-transitory computer-readable storage media (devices) and transmission media.

Non-transitory computer-readable storage media (devices) includes RAM, ROM, EEPROM, CD-ROM, solid state drives (SSDs) (e.g., based on RAM), Flash memory, phase-change memory (PCM), other types of memory, other optical disk storage, magnetic disk storage or other magnetic storage devices, or any other medium which can be used to store desired program code means in the form of computer-executable instructions or data structures and which can be accessed by a general purpose or special purpose computer.

A “network” is defined as one or more data links that enable the transport of electronic data between computer systems and/or modules and/or other electronic devices. When information is transferred or provided over a network or another communications connection (either hardwired, wireless, or a combination of hardwired or wireless) to a computer, the computer properly views the connection as a transmission medium. Transmissions media can include a network and/or data links which can be used to carry desired program code means in the form of computer-executable instructions or data structures and which can be accessed by a general purpose or special purpose computer. Combinations of the above should also be included within the scope of computer-readable media.

Further, upon reaching various computer system components, program code means in the form of computer-executable instructions or data structures can be transferred automatically from transmission media to non-transitory computer-readable storage media (devices) (or vice versa). For example, computer-executable instructions or data structures received over a network or data link can be buffered in RAM within a network interface module (e.g., a NIC), and then eventually transferred to computer system RAM and/or to less volatile computer storage media (devices) at a computer system. Thus, it should be understood that non-transitory computer-readable storage media (devices) can be included in computer system components that also (or even primarily) utilize transmission media.

Computer-executable instructions comprise, for example, instructions and data which, when executed at a processor, cause a general purpose computer, special purpose computer, or special purpose processing device to perform a certain function or group of functions. In some embodiments, computer-executable instructions are executed on a general-purpose computer to turn the general-purpose computer into a special purpose computer implementing elements of the disclosure. The computer executable instructions may be, for example, binaries, intermediate format instructions such as assembly language, or even source code. Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

Those skilled in the art will appreciate that the disclosure may be practiced in network computing environments with many types of computer system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, tablets, pagers, routers, switches, and the like. The disclosure may also be practiced in distributed system environments where local and remote computer systems, which are linked (either by hardwired data links, wireless data links, or by a combination of hardwired and wireless data links) through a network, both perform tasks. In a distributed system environment, program modules may be located in both local and remote memory storage devices.

Embodiments of the present disclosure can also be implemented in cloud computing environments. In this description, “cloud computing” is defined as a model for enabling on-demand network access to a shared pool of configurable computing resources. For example, cloud computing can be employed in the marketplace to offer ubiquitous and convenient on-demand access to the shared pool of configurable computing resources. The shared pool of configurable computing resources can be rapidly provisioned via virtualization and released with low management effort or service provider interaction, and then scaled accordingly.

A cloud-computing model can be composed of various characteristics such as, for example, on-demand self-service, broad network access, resource pooling, rapid elasticity, measured service, and so forth. A cloud-computing model can also expose various service models, such as, for example, Software as a Service (SaaS), Platform as a Service (PaaS), and Infrastructure as a Service (IaaS). A cloud-computing model can also be deployed using different deployment models such as private cloud, community cloud, public cloud, hybrid cloud, and so forth. In this description and in the claims, a “cloud-computing environment” is an environment in which cloud computing is employed.

FIG. 8 illustrates a block diagram of a computing device 800 that may be configured to perform one or more of the processes described above. One will appreciate that one or more computing devices such as the computing device 800 may implement the bubble-detection system 106 and the sequencing system 104. As shown by FIG. 8, the computing device 800 can comprise a processor 802, a memory 804, a storage device 806, an I/O interface 808, and a communication interface 810, which may be communicatively coupled by way of a communication infrastructure 812. In certain embodiments, the computing device 800 can include fewer or more components than those shown in FIG. 8. The following paragraphs describe components of the computing device 800 shown in FIG. 8 in additional detail.

In one or more embodiments, the processor 802 includes hardware for executing instructions, such as those making up a computer program. As an example, and not by way of limitation, to execute instructions for dynamically modifying workflows, the processor 802 may retrieve (or fetch) the instructions from an internal register, an internal cache, the memory 804, or the storage device 806 and decode and execute them. The memory 804 may be a volatile or non-volatile memory used for storing data, metadata, and programs for execution by the processor(s). The storage device 806 includes storage, such as a hard disk, flash disk drive, or other digital storage device, for storing data or instructions for performing the methods described herein.

The I/O interface 808 allows a user to provide input to, receive output from, and otherwise transfer data to and receive data from computing device 800. The I/O interface 808 may include a mouse, a keypad or a keyboard, a touch screen, a camera, an optical scanner, network interface, modem, other known I/O devices or a combination of such I/O interfaces. The I/O interface 808 may include one or more devices for presenting output to a user, including, but not limited to, a graphics engine, a display (e.g., a display screen), one or more output drivers (e.g., display drivers), one or more audio speakers, and one or more audio drivers. In certain embodiments, the I/O interface 808 is configured to provide graphical data to a display for presentation to a user. The graphical data may be representative of one or more graphical user interfaces and/or any other graphical content as may serve a particular implementation.

The communication interface 810 can include hardware, software, or both. In any event, the communication interface 810 can provide one or more interfaces for communication (such as, for example, packet-based communication) between the computing device 800 and one or more other computing devices or networks. As an example, and not by way of limitation, the communication interface 810 may include a network interface controller (NIC) or network adapter for communicating with an Ethernet or other wire-based network or a wireless NIC (WNIC) or wireless adapter for communicating with a wireless network, such as a WI-FI.

Additionally, the communication interface 810 may facilitate communications with various types of wired or wireless networks. The communication interface 810 may also facilitate communications using various communication protocols. The communication infrastructure 812 may also include hardware, software, or both that couples components of the computing device 800 to each other. For example, the communication interface 810 may use one or more networks and/or protocols to enable a plurality of computing devices connected by a particular infrastructure to communicate with each other to perform one or more aspects of the processes described herein. To illustrate, the sequencing process can allow a plurality of devices (e.g., a client device, sequencing device, and server device(s)) to exchange information such as sequencing data and error notifications.

In the foregoing specification, the present disclosure has been described with reference to specific exemplary embodiments thereof. Various embodiments and aspects of the present disclosure(s) are described with reference to details discussed herein, and the accompanying drawings illustrate the various embodiments. The description above and drawings are illustrative of the disclosure and are not to be construed as limiting the disclosure. Numerous specific details are described to provide a thorough understanding of various embodiments of the present disclosure.

The present disclosure may be embodied in other specific forms without departing from its spirit or essential characteristics. The described embodiments are to be considered in all respects only as illustrative and not restrictive. For example, the methods described herein may be performed with less or more steps/acts or the steps/acts may be performed in differing orders. Additionally, the steps/acts described herein may be repeated or performed in parallel with one another or in parallel with different instances of the same or similar steps/acts. The scope of the present application is, therefore, indicated by the appended claims rather than by the foregoing description. All changes that come within the meaning and range of equivalency of the claims are to be embraced within their scope.

What is claimed is:

1. A system comprising:
at least one processor; and
a non-transitory computer readable medium comprising instructions that, when executed by the at least one processor, cause the system to:
receive, for a nucleotide-sample slide, call data comprising nucleobase calls for cycles of sequencing a nucleic-acid polymer;
receive, for the nucleotide-sample slide, quality data comprising quality metrics that estimate errors in the nucleobase calls for the cycles;
determine, from the nucleobase calls for the cycles, a first subset of the nucleobase calls corresponding to at least one nucleobase and a second subset of the nucleobase calls satisfying a threshold quality metric for the quality metrics; and
detect a presence of a bubble within the nucleotide-sample slide utilizing a bubble-detection-machine-learning model based on the first subset of the nucleobase calls and the second subset of the nucleobase calls.

2. The system as recited in claim 1, further comprising instructions that, when executed by the at least one processor, cause the system to:
receive the call data and the quality data for a section of the nucleotide-sample slide; and
detect the presence of the bubble within the section of the nucleotide-sample slide.

3. The system as recited in claim 2, further comprising instructions that, when executed by the at least one processor, cause the system to detect the presence of the bubble within the section of the nucleotide-sample slide by detecting the bubble within a tile of a flow cell.

4. The system as recited in claim 1, further comprising instructions that, when executed by the at least one processor, cause the system to determine the first subset of the nucleobase calls corresponding to the at least one nucleobase by determining at least one of a subset of adenine calls, a subset of thymine calls, a subset of cytosine calls, or a subset of guanine calls for the cycles of sequencing the nucleic-acid polymer.

5. The system as recited in claim 4, further comprising instructions that, when executed by the at least one processor, cause the system to detect the presence of the bubble utilizing the bubble-detection-machine-learning model by extracting, utilizing layers of the bubble-detection-machine-learning model, features from an input matrix comprising the subset of adenine calls, the subset of guanine calls, and the second subset of the nucleobase calls satisfying the threshold quality metric for the cycles of sequencing the nucleic-acid polymer.

6. The system as recited in claim 1, further comprising instructions that, when executed by the at least one processor, cause the system to detect the presence of the bubble by detecting at least one of an air bubble, an oil bubble, or a ghost bubble within the nucleotide-sample slide.

7. The system as recited in claim 1, wherein the bubble-detection-machine-learning model comprises a convolutional neural network comprising feature extraction layers, classification layers, and an adaptive max pooling layer between the feature extraction layers and the classification layers.

8. The system as recited in claim 1, further comprising instructions that, when executed by the at least one processor, cause the system to detect the presence of the bubble by:
generating, utilizing the bubble-detection-machine-learning model, a probability that a section of the nucleotide-sample slide contains the bubble; and
determining that the probability satisfies a threshold value indicating the presence of the bubble.

9. The system as recited in claim 1, further comprising instructions that, when executed by the at least one processor, cause the system to receive the call data comprising the nucleobase calls based on:
one-channel data comprising a single image for each section of the nucleotide-sample slide for a given cycle of sequencing the nucleic-acid polymer;
two-channel data comprising two images for each section of the nucleotide-sample slide for the given cycle of sequencing the nucleic-acid polymer; or
four-channel data comprising four images for each section of the nucleotide-sample slide for the given cycle of sequencing the nucleic-acid polymer.

10. The system as recited in claim 1, further comprising instructions that, when executed by the at least one processor, cause the system to determine the presence of the bubble during one or more cycles of the cycles of sequencing the nucleic-acid polymer.

11. A non-transitory computer readable medium comprising instructions that, when executed by at least one processor, cause a computing device to:
receive, for a nucleotide-sample slide, call data comprising nucleobase calls for cycles of sequencing a nucleic-acid polymer;
receive, for the nucleotide-sample slide, quality data comprising quality metrics that estimate errors in the nucleobase calls for the cycles;
determine, from the nucleobase calls for the cycles, a first subset of the nucleobase calls corresponding to at least one nucleobase and a second subset of the nucleobase calls satisfying a threshold quality metric for the quality metrics; and
detect a presence of a bubble within the nucleotide-sample slide utilizing a bubble-detection-machine-learning model based on the first subset of the nucleobase calls and the second subset of the nucleobase calls.

12. The non-transitory computer readable medium as recited in claim 11, wherein the bubble-detection-machine-learning model comprises at least one of a Support Vector Machine or an Adaptive Boosting machine learning model.

13. The non-transitory computer readable medium as recited in claim 11, further comprising instructions that, when executed by the at least one processor, cause the computing device to, based on detecting the presence of the bubble, provide, for display on the computing device, an alert indicating the presence of the bubble within the nucleotide-sample slide.

14. The non-transitory computer readable medium as recited in claim 11, further comprising instructions that, when executed by the at least one processor, cause the computing device to:
receive the call data and the quality data for a section of the nucleotide-sample slide; and
detect the presence of the bubble within the section of the nucleotide-sample slide.

15. The non-transitory computer readable medium as recited in claim 14, further comprising instructions that, when executed by the at least one processor, cause the computing device to detect the presence of the bubble within the section of the nucleotide-sample slide by detecting the bubble within a tile of a flow cell.

16. The non-transitory computer readable medium as recited in claim 11, further comprising instructions that, when executed by the at least one processor, cause the computing device to determine the presence of the bubble during a cycle of the cycles of sequencing the nucleic-acid polymer.

17. A computer-implemented method comprising:
receiving, for a nucleotide-sample slide, call data comprising nucleobase calls for cycles of sequencing a nucleic-acid polymer;
receiving, for the nucleotide-sample slide, quality data comprising quality metrics that estimate errors in the nucleobase calls for the cycles;
determining, from the nucleobase calls for the cycles, a first subset of the nucleobase calls corresponding to at least one nucleobase and a second subset of the nucleobase calls satisfying a threshold quality metric for the quality metrics; and
detecting a presence of a bubble within the nucleotide-sample slide utilizing a bubble-detection-machine-learning model based on the first subset of the nucleobase calls and the second subset of the nucleobase calls.

18. The computer-implemented method as recited in claim 17, wherein determining the first subset of the nucleobase calls corresponding to the at least one nucleobase comprises determining at least one of a subset of adenine calls, a subset of thymine calls, a subset of cytosine calls, or a subset of guanine calls for the cycles of sequencing the nucleic-acid polymer.

19. The computer-implemented method as recited in claim 17, further comprising modifying a quality metric for a nucleobase call based on detecting the presence of the bubble utilizing the bubble-detection-machine-learning model.

20. The computer-implemented method as recited in claim 17, wherein detecting the presence of the bubble comprises detecting at least one of an air bubble, an oil bubble, or a ghost bubble within the nucleotide-sample slide.
